(19) United States
(12) Patent Application Publication
(10) Pub. No.: US 2014/0147874 A1
(43) Pub. Date: May 29, 2014

(54) BIOMARKERS OF CARDIAC ISCHEMIA

(71) Applicant: Catherine Cotter, Baltimore, MD (US)

(72) Inventors: Robert J. Cotter, Baltimore, MD (US); Christine Jelinek, Baltimore, MD (US); Jennifer E. Van Eyk, Baltimore, MD (US)

(73) Assignee: The Johns Hopkins University, Baltimore, MD (US)

(21) Appl. No.: 14/003,078

(22) PCT Filed: Mar. 5, 2012
(86) PCT No.: PCT/US2012/027711

§ 371 Date: Nov. 15, 2013

Related U.S. Application Data
(60) Provisional application No. 61/449,306, filed on Mar. 4, 2011.

(51) Int. Cl. C12Q 1/34

(52) U.S. Cl. CPC C12Q 1/34
USPC 435/18

(57) ABSTRACT

The present invention relates to the field of biomarkers. More specifically, the present invention relates to biomarkers useful in diagnosing cardiac ischemia. In a specific embodiment, a method for diagnosing acute cardiac ischemia in a patient comprises the steps of (a) measuring the levels of one or more post-translationally modified and unmodified serum albumin peptides in a sample collected from the patient using SRM-MS, wherein the post-translationally modified peptides are phosphorylated and/or cysteinylated; (b) comparing the levels of the measured one or more post-translationally modified serum albumin peptides to the levels of the measured one or more unmodified serum albumin peptides; and (c) correlating the compared levels to a patient having ACI or to a patient not having ACI, thereby providing the diagnosis.

### CROSS-REFERENCE TO RELATED APPLICATIONS

This application claims the benefit of U.S. Provisional Application No. 61/449,306, filed Mar. 4, 2011, which is incorporated herein by reference in its entirety.

### STATEMENT OF GOVERNMENTAL INTEREST

This invention was made with U.S. government support under grant no. HHSN268201000032C. The U.S. government has certain rights in the invention.

### FIELD OF THE INVENTION

The present invention relates to the field of biomarkers. More specifically, the present invention relates to biomarkers useful in diagnosing cardiac ischemia.

### INCORPORATION-BY-REFERENCE OF MATERIAL SUBMITTED ELECTRONICALLY

This application contains a sequence listing. It has been submitted electronically via EFS-Web as an ASCII text file entitled “P11425-02_ST25.txt.” The sequence listing is 19,046 bytes in size, and was created on Mar. 1, 2012. It is hereby incorporated by reference in its entirety.

### BACKGROUND OF THE INVENTION

In ischemic heart disease (IHD), prolonged oxygen deprivation to cardiomyocytes—acute cardiac ischemia (ACI), caused by occlusion of a coronary artery induces cellular damage and/or necrosis—cute myocardial infarction (AMI). IHD remains a major cause of US hospital emergency department admissions and AMI continues to be the most common cause of death in first world countries. The urgency with which care must be administered to a patient with IHD is ultimately what makes the disease so deadly. In fact, the survival rate of a patient suffering from ACI is largely determined within the first several hours of hospital admission. Thus the speed in which a patient can be conclusively diagnosed with ACI and properly triaged is often the critical determinant of a patient's overall outcome.

Available emergency treatments such as thrombolytic therapy or acute angioplasty are very successful in reversing coronary occlusions in an emergency department setting. If either is administered within four hours of the onset of ACI symptoms, the degree of myocardial tissue damage that the patient sustains is greatly reduced, increasing the patient's chance of survival exponentially. However, diagnosing ACI patients represents a serious bottleneck in IHD patient care. ACI symptoms are often subtle, vague, and varied across the population. As such, patient assessment using ischemic symptoms lacks the rigor needed for a conclusive clinical diagnosis and cannot be used to direct patient treatment. Chest pains (the most common symptom associated with IHD) can be the by-product of any number of medical conditions; a patient with a benign non-cardiac disorder will present to the emergency department almost identically as a patient with a potentially life-threatening AMI. The current gold standard in ACI diagnostics—the EKG—lacks some sensitivity and specificity, can lead to missed diagnoses, and requires a skilled interpreter; less experienced emergency department interpreters operate at lower positive predictive values than trained interpreters. As any delay between patient admission and positive diagnosis greatly impacts a patient's mortality risk, developing a blood-based ischemic biomarker assay capable of generating a diagnostic outcome prior to irreversible myocardial necrosis is often heralded as a “Holy Grail.”

### SUMMARY OF THE INVENTION

The present invention is based, at least in part, on the discovery that several post-translational modifications (PTMs) of human serum albumin, specifically, phosphorylation and cysteinylation, correlate to cardiac ischemia prior to myocardial infarction (MI). The amino acid sequence for human serum albumin is shown in SEQ ID NO:1. SEQ ID NOS: 2-4 represent the amino acid sequences for Domain I, Domain II and Domain III of human serum albumin, respectively. Accordingly, these PTMs can function as biomarkers for pre-ischemia change. Selected reaction monitoring mass spectrometry (SRM-MS) is used as a method to quantify the PTMs, although other analytical platforms such as ELISA or other immunoassays can be used. As described herein, the present inventors have created a rapid, highly specific and sensitive multiplex SRM-MS biomarker assay for cardiac ischemia.

Accordingly, in one aspect, the present invention provides methods for diagnosing cardiac ischemia. In one embodiment, a method for diagnosing acute cardiac ischemia (ACI) in a patient comprises the steps of (a) measuring the levels of one or more serum albumin biomarker peptides in a sample collected from the patient using selected reaction monitoring mass spectrometry (SRM-MS); and (b) comparing the levels of the one or more biomarkers with predefined levels of the same biomarkers that correlate to a patient having ACI and predefined levels of the same biomarkers that correlate to a patient not having ACI, wherein a correlation to one of the predefined levels provides the diagnosis.

In a specific embodiment, the one or more serum albumin biomarker peptides is selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12. In a more specific embodiment, the one or more serum albumin biomarker peptides comprises SEQ ID NO:5, SEQ ID NO:6, SEQ ID NO:7, SEQ ID NO:8, SEQ ID NO:9, SEQ ID NO:10, SEQ ID NO:11 and SEQ ID NO:12. In yet another embodiment, the one or more serum albumin biomarker peptides further comprises one or more serum albumin biomarker peptides selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28. See Table 1, infra.

In a further embodiment, the one or more serum albumin biomarker peptides is selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12, SEQ ID NO:13, SEQ ID NO:14, SEQ ID NO:15, SEQ ID NO:16, SEQ ID NO:17. SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

In another embodiment, the one or more serum albumin biomarker peptides comprises SEQ ID NO:5, SEQ ID NO:6, SEQ ID NO:7, SEQ ID NO:8, SEQ ID NO:9, SEQ ID NO:10, SEQ ID NO:11 and SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

In a specific embodiment, the one or more serum albumin biomarker peptides comprises SEQ ID NO:5. In another embodiment, the one or more serum albumin biomarker peptides comprises SEQ ID NO:6. In yet another embodiment, the one or more serum albumin biomarker peptides comprises SEQ ID NO:7. The one or more serum albumin biomarker peptides may comprise SEQ ID NO:8. The one or more serum albumin biomarker peptides may instead comprise SEQ ID NO:9. In another specific embodiment, the one or more serum albumin biomarker peptides comprises SEQ ID NO:10. In a further embodiment, the one or more serum albumin biomarker peptides comprises SEQ ID NO:11. In yet another embodiment, the one or more serum albumin biomarker peptides comprises SEQ ID NO:12.

The patient sample may comprise a blood, plasma, or serum sample. In a specific embodiment, the sample is a blood sample. In another embodiment, the patient sample is a plasma sample. In yet another embodiment, the sample is a serum sample.

Alternatively, a method for diagnosing ACI in a patient comprises the steps of (a) collecting a sample from the patient: (b) measuring the levels of a panel of serum albumin biomarker peptides in the sample collected from the patient using SRM-MS, wherein the panel of biomarkers comprises SEQ ID NO:5, SEQ ID NO:6, SEQ ID NO:7, SEQ ID NO:8. SEQ ID NO:9, SEQ ID NO:10, SEQ ID NO:11 and SEQ ID NO:12; and (c) comparing the levels of the panel of biomarkers with predefined levels of the same panel of biomarkers that correlate to a patient having ACI and predefined levels of the same panel of biomarkers that correlate to a patient not having ACI, wherein a correlation to one of the predefined levels provides the diagnosis. The panel of biomarkers can further comprises one or more serum albumin biomarker peptides selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28

In another embodiment, a method for determining the ACI status in a patient comprises the steps of (a) collecting a sample from the patient; (b) measuring the levels of a panel of serum albumin biomarker peptides in the sample collected from the patient using SRM-MS, wherein the panel of biomarkers comprises SEQ ID NO:5, SEQ ID NO:6, SEQ ID NO:7, SEQ ID NO:8, SEQ ID NO:9, SEQ ID NO:10, SEQ ID NO:11 and SEQ ID NO:12; and (c) comparing the levels of the panel of biomarkers with predefined levels of the same panel of biomarkers that correlate to one or more ACI statuses selected from the group consisting of having ACI, not having ACI, progressing ACI, and regressing ACI, wherein a correlation to one of the predefined levels determines the ACI status of the patient. In a further embodiment, the panel of biomarkers further comprises one or more serum albumin biomarker peptides selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

In an alternative embodiment, a method for determining the ACI status in a patient comprises the steps of (a) collecting a sample from the patient, (b) measuring the levels of a panel of serum albumin biomarker peptides in the sample collected from the patient using SRM-MS, wherein the panel of biomarkers comprises one or more modified and/or unmodified peptides from Domain I of serum albumin; and (c) comparing the levels of the panel of biomarkers with predefined levels of the same panel of biomarkers that correlate to one or more ACI statuses selected from the group consisting of having ACI, not having ACI, progressing ACI, and regressing ACI, wherein a correlation to one of the predefined levels determines the ACI status of the patient. The one or more peptides from Domain I of serum albumin can be selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12. In another embodiment, the panel of biomarkers further comprises one or more modified and/or unmodified peptides from Domain II of serum albumin. In a specific embodiment, the one or more peptides from Domain I of serum albumin is selected from the group consisting of SEQ ID NO:21, SEQ ID NO:22, and SEQ ID NO:23. In yet another embodiment, the panel of biomarkers further comprises one or more modified and/or unmodified peptides from Domain III of serum albumin. Specifically, the one or more peptides from Domain I of serum albumin is selected from the group consisting of SEQ ID NO:24, SEQ ID NO:25, SEQ ID NO:26, SEQ ID NO:27, and SEQ ID NO:28.

In another aspect, the present invention provides diagnostic kits for diagnosing cardiac ischemia. In a specific embodiment, a diagnostic kit for diagnosing ACI in a patient comprises (a) a substrate for collecting a biological sample from the patient; and (b) means for measuring the levels of one or more human serum albumin biomarker peptides selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12. The one or more human serum albumin biomarker peptides can further comprise SEQ ID NO:13, SEQ ID NO:14, SEQ ID NO:15, SEQ ID NO:16. SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26. SEQ ID NO:27, and SEQ ID NO:28.

In another embodiment, the present invention provides a use of a peptide from domain I of human serum albumin in the manufacture of an assay for diagnosing acute cardiac ischemia (ACI) in a subject comprising (a) a peptide for measuring within a subject sample using selected reaction monitoring mass spectrometry (SRM-MS); and (b) comparing the measured peptide level with predefined levels of the same peptide that correlates to a patient having ACI and predefined levels of the same peptide that correlates to a patient not having ACI, wherein a correlation to one of the predefined levels provides the diagnosis. The peptide can be is selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12. The peptide can further comprise one or more peptides selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

In a more specific embodiment, a method for diagnosing ACI in a patient can comprise the steps of (a) measuring the levels of one or more modified and/or unmodified serum albumin proteins in a sample collected from the patient, wherein the modified proteins are phosphorylated and/or cysteinylated; and (b) comparing the levels of the measured one or more modified and/or unmodified serum albumin proteins with predefined levels of the same proteins that correlate to a patient having ACI and predefined levels of the same proteins that correlate to a patient not having ACI, wherein a correlation to one of the predefined levels provides the diagnosis.

In another embodiment, a method for diagnosing ACI in a patient comprises the steps of (a) measuring the levels of one or more post-translationally modified and unmodified serum albumin peptides in a sample collected from the patient using SRM-MS, wherein the post-translationally modified peptides are phosphorylated and/or cysteinylated; (b) comparing the levels of the measured one or more post-translationally modified serum albumin peptides to the levels of the measured one or more unmodified serum albumin peptides; and (c) correlating the compared levels to a patient having ACI or to a patient not having ACI, thereby providing the diagnosis. In other words, the comparison step could be the ratio of one biomarker to another, for example a phosphorylated and non-phosphorylated peptide.

In yet another embodiment, a method for diagnosing ACI in a patient comprises the steps of (a) measuring the levels of one or more post-translationally modified and unmodified serum albumin peptides in a sample collected from the patient using SRM-MS, wherein the post-translationally modified peptides are phosphorylated and/or cysteinylated; (b) comparing the ratio of the measured one or more post-translationally modified serum albumin peptides and the measured one or more unmodified serum albumin peptides to the ratio of one or more modified/unmodified serum albumin peptides; and (c) correlating the compared levels to a patient having ACI or to a patient not having ACI, thereby providing the diagnosis. For example the ratio of a phosphorylated to a non-phosphorylated peptide could be compared to the ratio of another modified/unmodified peptide.

### BRIEF DESCRIPTION OF THE FIGURES

FIG. 1 presents the schema of automated sample preparation and liquid chromatography (LC) separation performed for all experiments using the Perfinity Workstation.

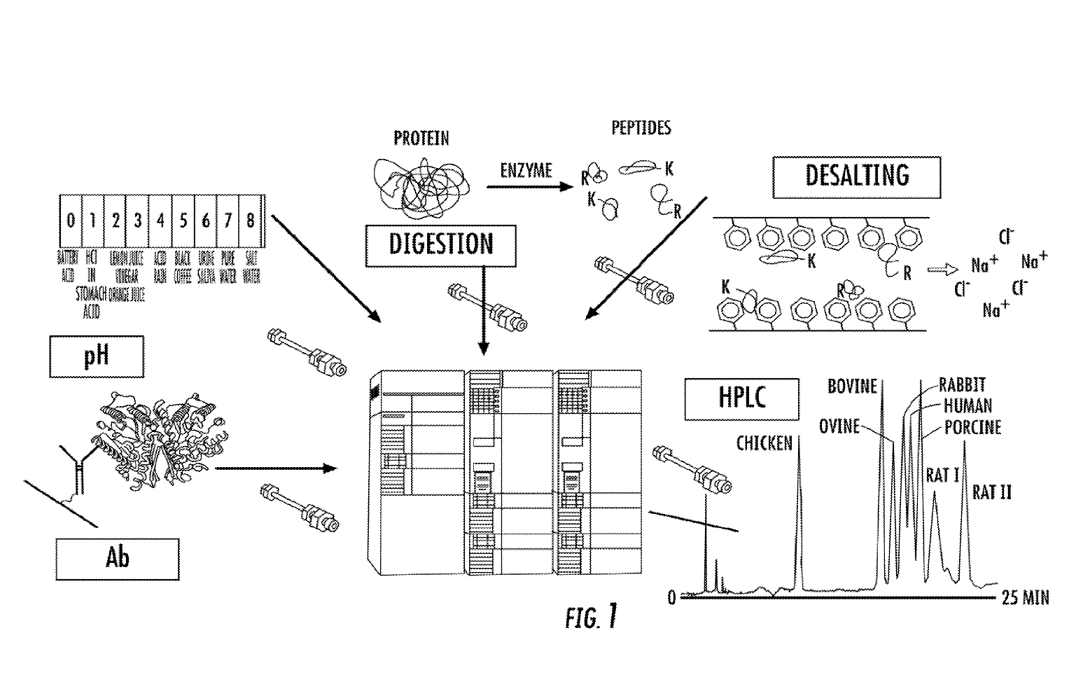

FIG. 2 is a graph showing that significant gains in reproducibility are achieved by automating sample preparation.

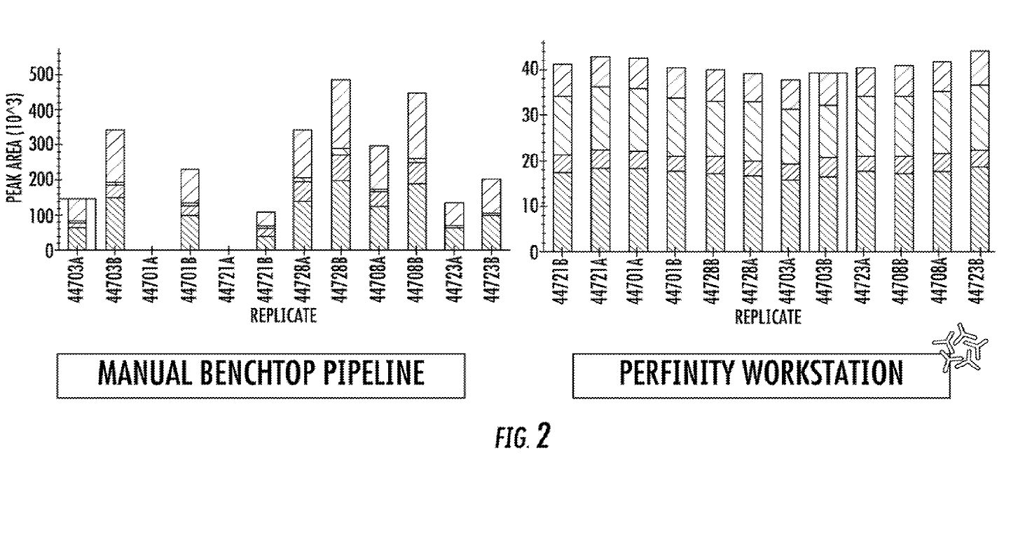

FIG. 3 presents the optimized workflow of a 35 minute assay from patient sample to mass spectra.

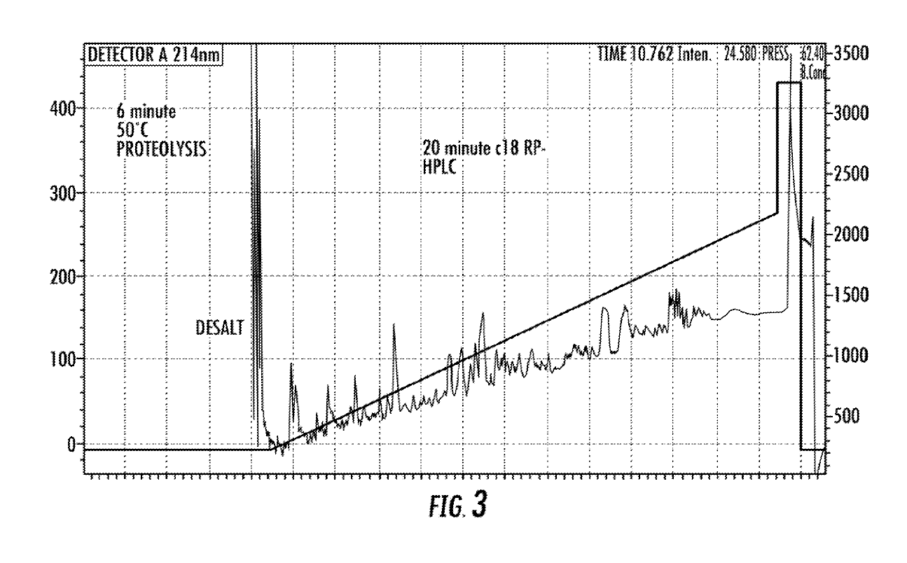

FIG. 4 presents the chromatographic peptide maps of five individual replicates using sample preparation performed in the Perfinity Workstation.

FIG. 5A shows a representative selected reaction monitoring (SRM) spectrum for the peptide GDFQFNISR (SEQ ID NO:29) from E. coli beta-galactosidase. Precursor: 542.2645++, Product Ions: [y5]−636.3464+[y4]−489.2780+ and [y2]−262.1510+. FIG. 5B is a standard curve for serial dilutions of E. coli beta-galactosidase in sera.

FIG. 6 presents the iterative method development approach used to design the SRM-MS assay.

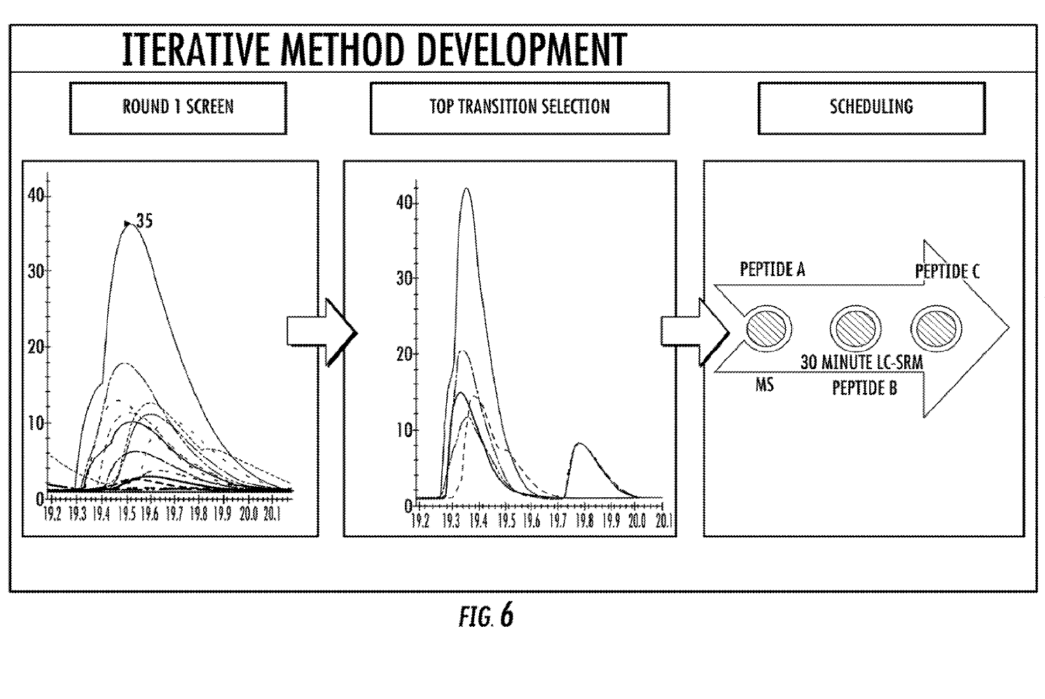

FIG. 7 shows retention time reproducibility. Each surrogate peptide is represented on the x-axis by one letter abbreviations of its first three amino acid residues. The transitions for each peptide are represented in different colors, and the vertical size of each point shows the range of retention times for triplicate analyses.

FIG. 8 is a table listing the eight peptides used in the SRM-MS assay.

FIG. 9 is a high resolution MS/MS fragment spectra for the human serum albumin PTM peptide TCVADESAENCDK (underlined indicates cysteinylation) (SEQ ID NO:9).

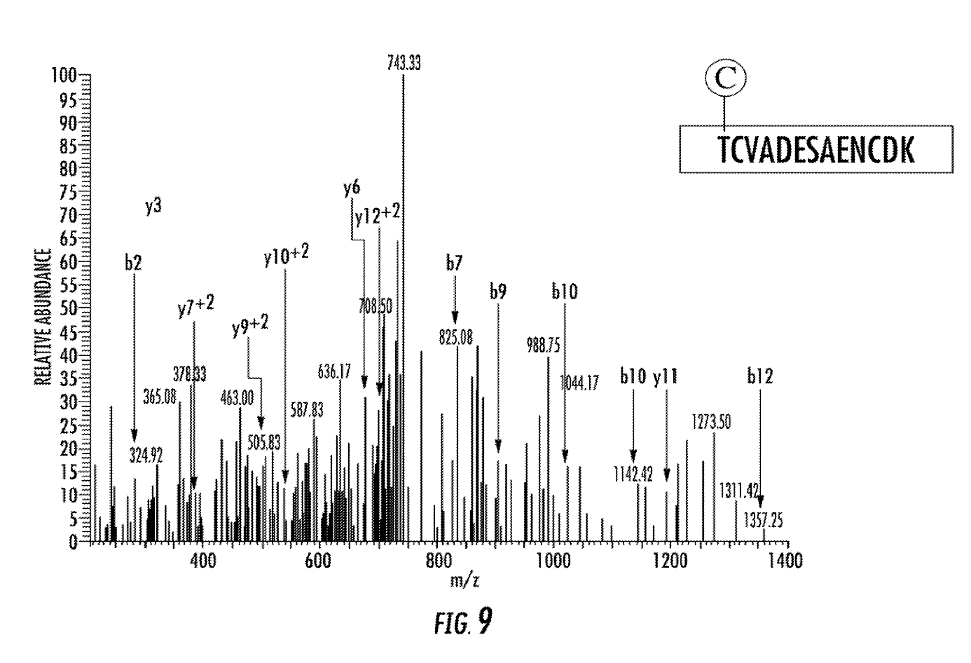

FIG. 10 is a high resolution MS/MS fragment spectra for the human serum albumin PTM peptide TCVADESAENCDK (underlined indicates phosphorylation) (SEQ ID NO:10).

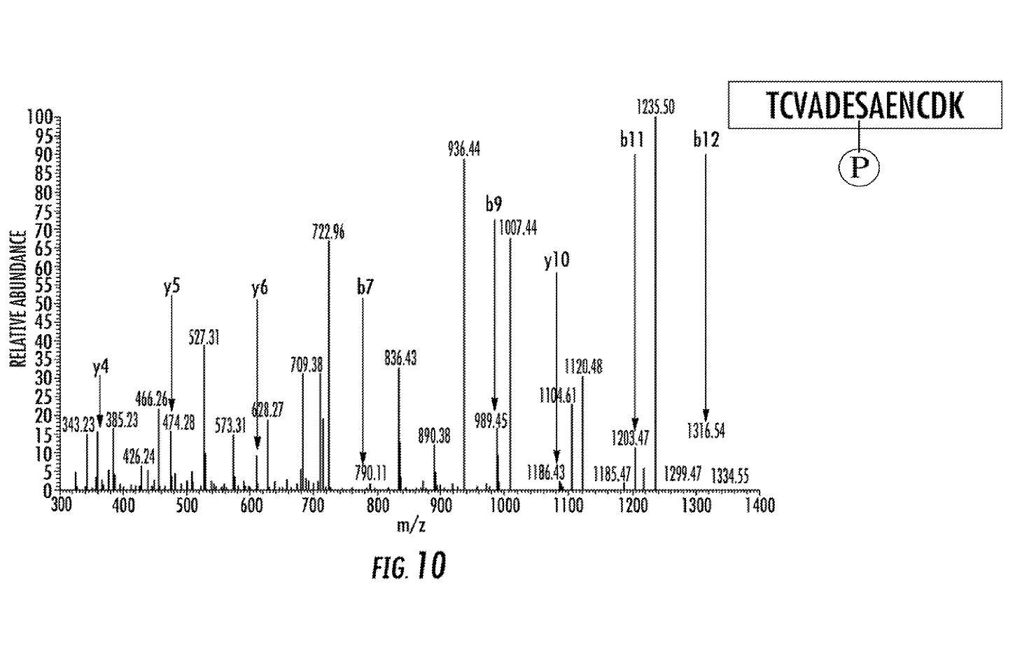

FIG. 11 is a high resolution MS/MS fragment spectra for the human serum albumin PTM peptide ALVLIAFAQYLQQCPFEDHVK (underlined indicates cysteinylation) (SEQ ID NO:12).

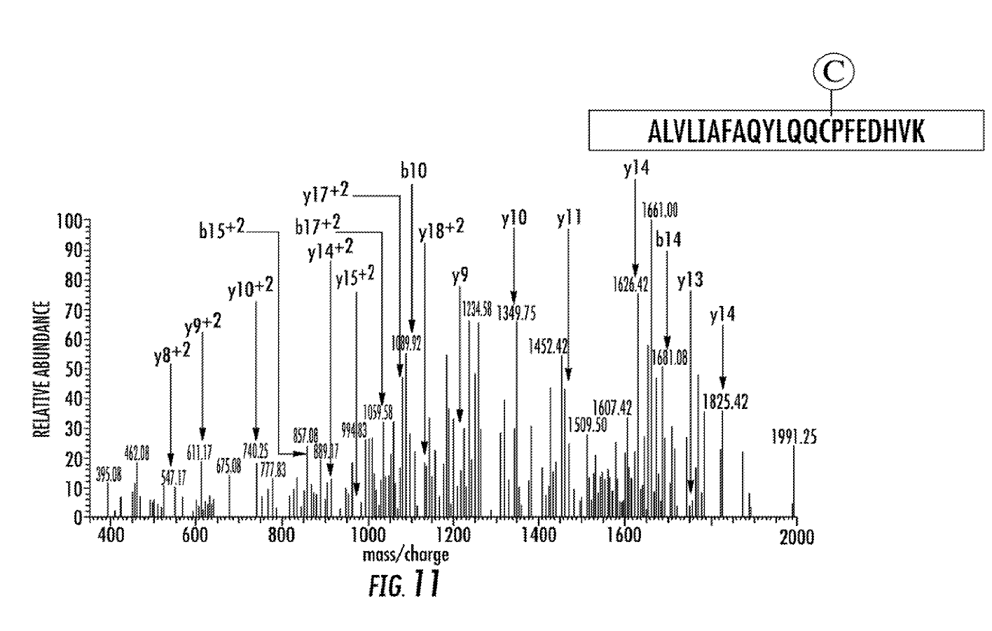

FIG. 12 is a high resolution MS/MS fragment spectra for the human serum albumin PTM peptide ETYGEMADCCAK (underlined indicates phosphorylation) (SEQ ID NO:7).

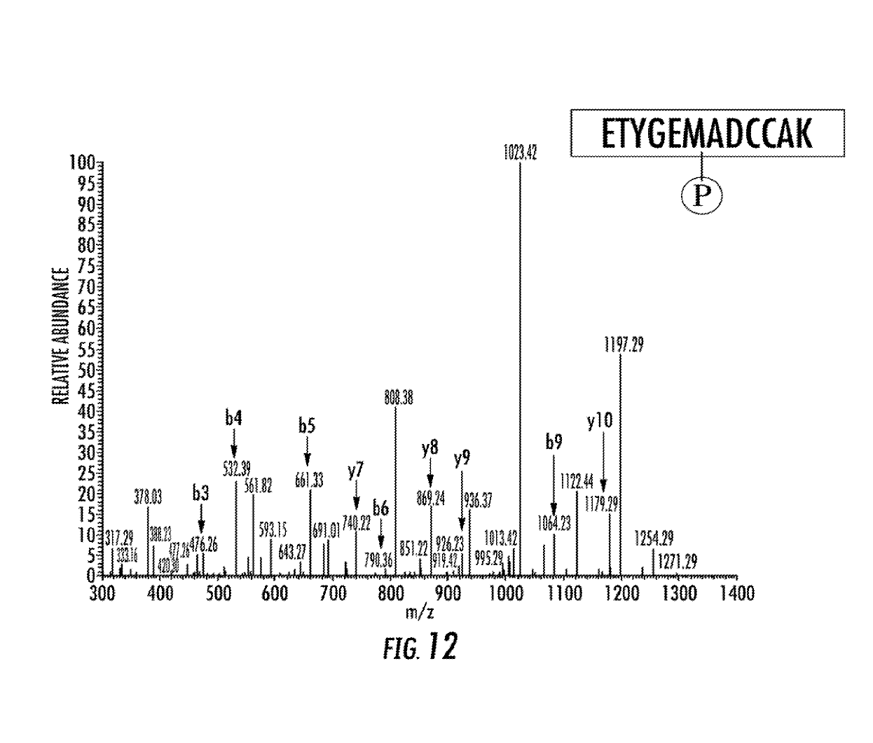

FIG. 13 shows the peptide SRM ion chromatograms for the human serum albumin PTM peptide ETYGEMADCCAK (phosphorylated (SEQ ID NO:7), unmodified (SEQ ID NO:5) and cysteinylated (SEQ ID NO:6)).

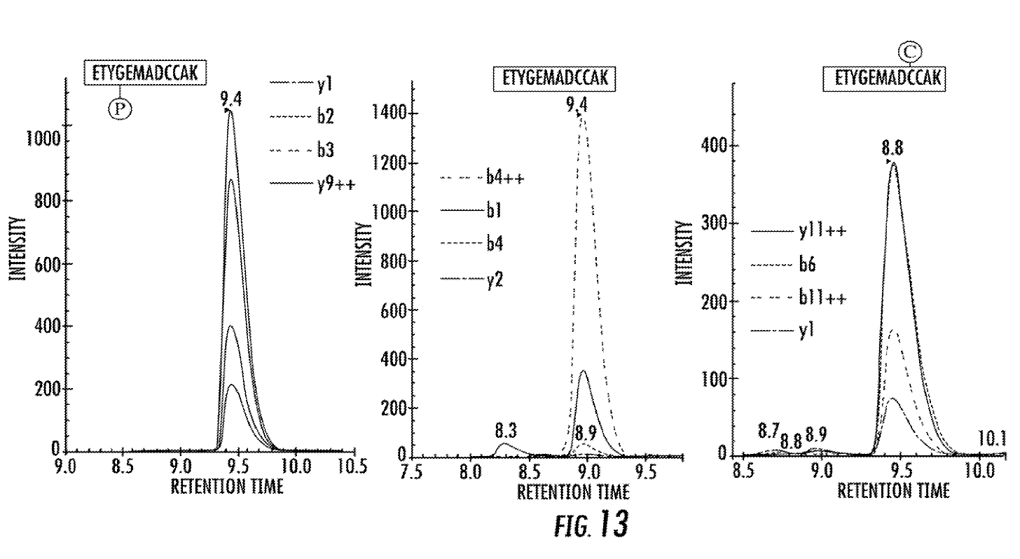

FIG. 14 shows the peptide SRM ion chromatograms for the human serum albumin PTM peptide TCVADESAENCDK (phosphorylated (SEQ ID NO:10), unmodified (SEQ ID NO:8) and cysteinylated (SEQ ID NO:9)).

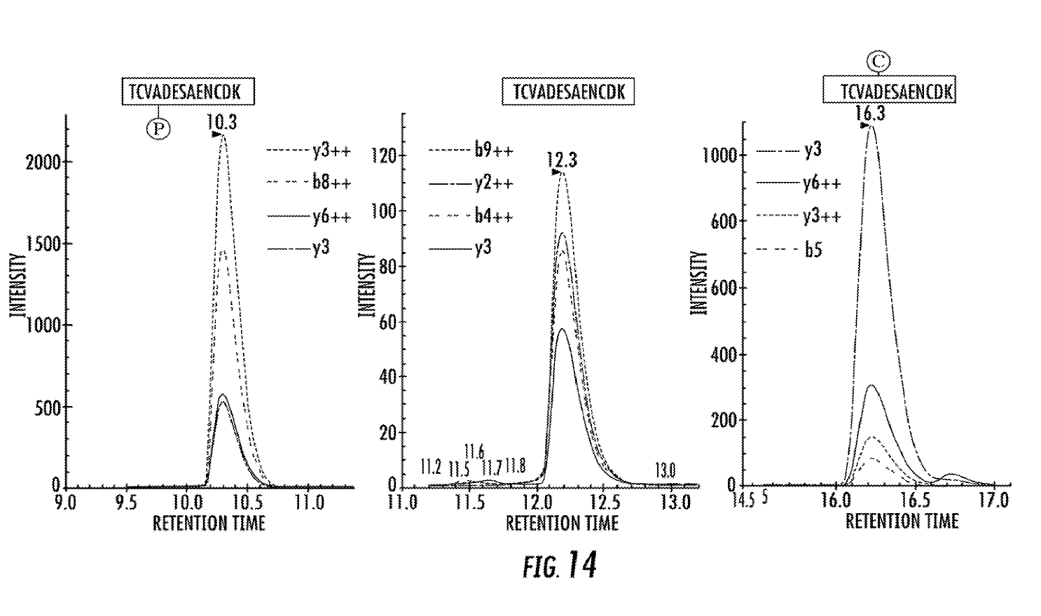

FIG. 15 shows the peptide SRM ion chromatograms for the human serum albumin PTM peptide ALVLIAFAQYLQQCPFEDHVK (unmodified (SEQ ID NO:11) and cysteinylated (SEQ ID NO:12)).

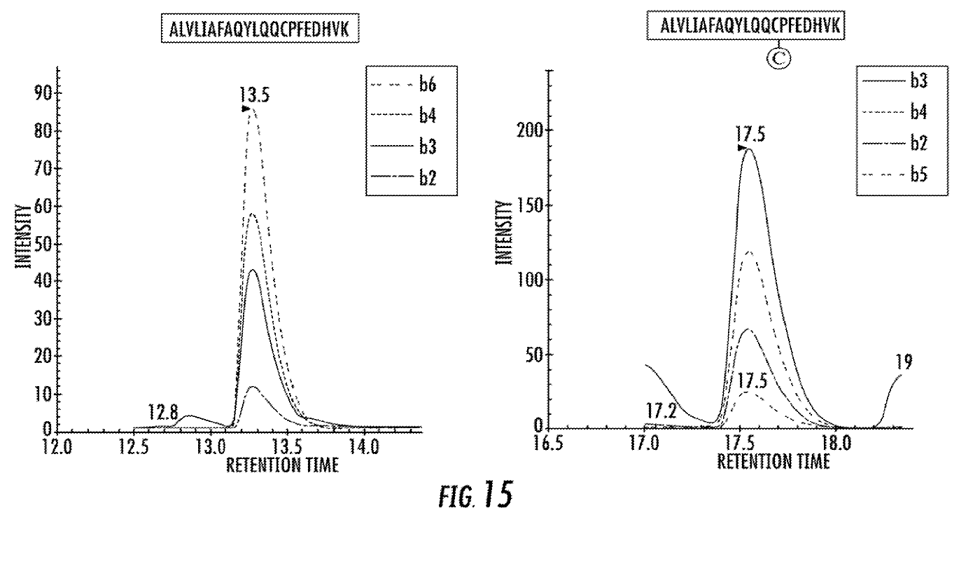

FIG. 16 is a table showing the statistical analysis from the SRM-MS assay results.

FIG. 17 shows the statistical analysis of gender bias assessment results from 30 control subjects for the human serum albumin PTM peptide TCVADESAENCDK (phosphorylated (SEQ ID NO:10), unmodified (SEQ ID NO:8) and cysteinylated (SEQ ID NO:9)).

FIG. 18 shows the statistical analysis of gender bias assessment results from 30 control subjects for the human serum albumin PTM peptide ETYGEMADCCAK (phosphorylated (SEQ ID NO:7), unmodified (SEQ ID NO:5) and cysteinylated (SEQ ID NO:6)).

FIG. 19 shows the statistical analysis of gender bias assessment results from 30 control subjects for the human serum albumin PTM peptide ALVLIAFAQYLQQCPFEDHVK (unmodified (SEQ ID NO:11) and cysteinylated (SEQ ID NO:12)).

FIG. 20 is a graph showing peptide chromatographic maps from individual male and female subjects.

FIG. 21 is a graph showing SRM-MS results from the induced ischemia cohort screen for the human serum albumin PTM peptide ALVLIAFAQYLQQCPFEDHVK (unmodified (SEQ ID NO:11)).

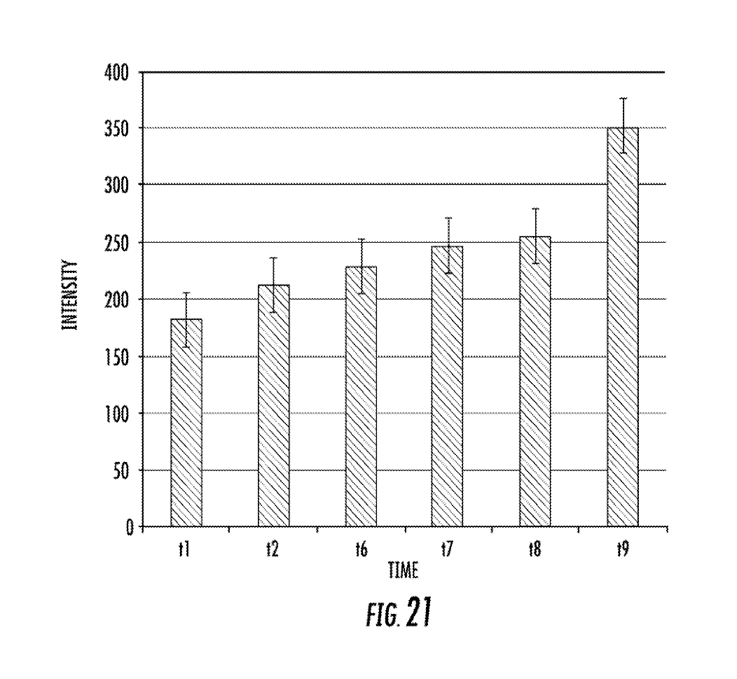

FIG. 22 is a graph showing SRM-MS results from the induced ischemia cohort screen for the human serum albumin PTM peptide TCVADESAENCDK (phosphorylated (SEQ ID NO:10)).

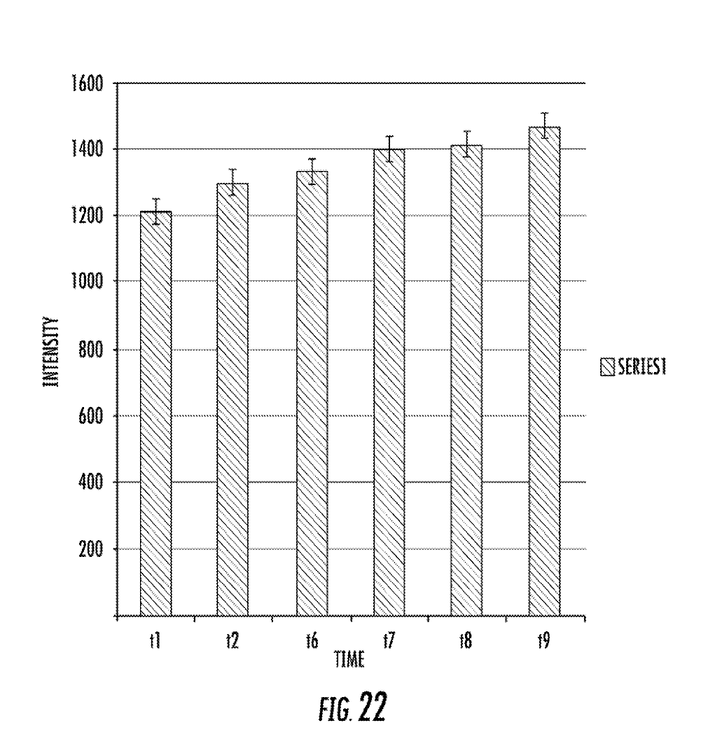

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Acute cardiac ischemia (ACI) occurs when myocardiacytes become oxygen deprived after cellular blood supply has been restricted or severed. If blood circulation is not restored, prolonged oxygen and nutrient starvation induces damage and/or necrosis to the affected myocardiacyte cells. This cell death is defined medically as an acute myocardial infarction (AMI). Treatment of ACI prior to widespread myocardium damage greatly enhances a patient's chances for survival. As such, early diagnosis and treatment is critical for effective therapeutic intervention. Unfortunately, there is an absence of effective diagnostic markers for ACI and the rapid progression of ischemic heart disease (IHD) makes developing a compatible assay platform a significant technological challenge.

### I. DEFINITIONS

As used herein, the term “comparing” refers to making an assessment of how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels that correspond to, for example, a patient having acute cardiac ischemia (ACI), not having ACI, is responding to treatment for ACI, is not responding to treatment for ACI, is/is not likely to respond to a particular ACI treatment, or having/not having another disease or condition. In a specific embodiment, the term “comparing” refers to assessing whether the level of one or more biomarkers of the present invention in a sample from a patient is the same as, more or less than, different from other otherwise correspond (or not) to levels of the same biomarkers in a control sample (e.g., predefined levels that correlate to uninfected individuals, standard ACI levels, etc.).

In another embodiment, the term “comparing” refers to making an assessment of how the proportion, level or cellular localization of one or more biomarkers in a sample from a patient relates to the proportion, level or cellular localization of the corresponding one or more biomarkers in the same sample. For example, a ratio of one biomarker to another from the same patient sample can be compared. In a specific embodiment, the proportion of a phosphorylated human serum albumin PTM can be compared to the unmodified PTM, both of which are measured in the same patient sample.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has ACI. In specific embodiments, the parameter may comprise the level of one or more biomarkers of the present invention. A particular set or pattern of the amounts of one or more biomarkers may indicate that a patient has ACI (i.e., correlates to a patient having ACI). In specific embodiments, a correlation could be the ratio of a phosphorylated peptide to the non-phosphorylated form, or any other combination in which a change in one peptide causes or is accompanied by a change in another. In other embodiments, a particular set or pattern of the amounts of one or more biomarkers may be correlated to a patient being unaffected (i.e., indicates a patient does not have ACI). In certain embodiments, “indicating,” or “correlating,” as used according to the present invention, may be by any linear or non-linear method of quantifying the relationship between levels of biomarkers to a standard, control or comparative value for the assessment of the diagnosis, prediction of ACI or ACI progression, assessment of efficacy of clinical treatment, identification of a patient that may respond to a particular treatment regime or pharmaceutical agent, monitoring of the progress of treatment, and in the context of a screening assay, for the identification of an anti-ACI therapeutic.

The terms “patient,” “individual,” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout, and refer to methods which include obtaining a patient sample and/or detecting the level of a biomarker(s) in a sample. In one embodiment, the terms refer to obtaining a patient sample and detecting the level of one or more biomarkers in the sample. In another embodiment, the terms “measuring” and “determining” mean detecting the level of one or more biomarkers in a patient sample. Measuring can be accomplished by methods known in the art and those further described herein. The term “measuring” is also used interchangeably throughout with the term “detecting.”

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of ACI. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cerebrospinal fluid, urine, saliva, stool and synovial fluid), solid tissue samples such as a biopsy specimen or tissue cultures or cells derived therefrom and the progeny thereof. In a specific embodiment, a sample comprises a blood sample. In another embodiment, a sample comprises a plasma sample. In yet another embodiment, a serum sample is used. The definition also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed for levels in a sample from an unaffected individual (UI) or a normal control individual (NC) (both terms are used interchangeably herein). In another embodiment, a “suitable control” or “appropriate control” is a value, level, feature, characteristic, property, etc. determined prior to performing a therapy (e.g., an ACI treatment) on a patient. In yet another embodiment, a transcription rate, mRNA level, translation rate, protein level, biological activity, cellular characteristic or property, genotype, phenotype, etc., can be determined prior to, during, or after administering a therapy into a cell, organ, or patient. In a further embodiment, a “suitable control” or “appropriate control” is a predefined value, level, feature, characteristic, property, etc. A “suitable control” can be a profile or pattern of levels of one or more biomarkers of the present invention that correlates to ACI, to which a patient sample can be compared. The patient sample can also be compared to a negative control, i.e., a profile that correlates to not having ACI.

### II. DETECTION OF CARDIAC ISCHEMIA BIOMARKERS

A. Detection by Mass Spectrometry

In one aspect, the biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, the biomarkers of the present invention are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic coelution of multiple transitions for a given analyte. The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application the terms SRM and MRM or also SRM/MRM can be used interchangeably, since they both refer to the same mass spectrometer operating principle. As a matter of clarity, the term SRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein. In another embodiment, the mass spectrometric technique is multiple reaction monitoring (MRM) or quantitative MRM.

In an alternative embodiment, the mass spectrometric technique comprises surface enhanced laser desorption and ionization or “SELDI,” as described, for example, in U.S. Pat. No. 6,225,047 and No. 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

B. Detection by Immunoassay

In other embodiments, the biomarkers of the present invention can be detected and/or measured by immunoassay. Immunoassay requires biospecific capture reagents, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art.

The present invention contemplates traditional immunoassays including, for example, sandwich immunoassays including ELISA or fluorescence-based immunoassays, immunoblots. Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds all neurogranin and/or one or more of its breakdown products might be used. Aptamers are nucleic acid-based molecules that bind specific ligands. Methods for making aptamers with a particular binding specificity are known as detailed in U.S. Pat. No. 5,475,096; No. 5,670,637; No. 5,696,249; No. 5,270,163; No. 5,707,796; No. 5,595,877; No. 5,660,985; No. 5,567,588; No. 5,683,867; No. 5,637,459; and No. 6,011,020.

C. Detection by Electrochemicaluminescent Assay

In several embodiments, the biomarker biomarkers of the present invention may be detected by means of an electrochcmicaluminescent assay developed by Meso Scale Discovery (Gaithersburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,997; No. 7,491,540; No. 7,288,410; No. 7,036,946; No. 7,052,861; No. 6,977,722; No. 6,919,173; No. 6,673,533; No. 6,413,783; No. 6,362,011; No. 6,319,670; No. 6,207,369; No. 6,140,045; No. 6,090,545; and No. 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

D. Other Methods for Detecting Biomarkers

The biomarkers of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

### III. DETERMINATION OF A PATIENT'S ACI STATUS

The present invention relates to the use of biomarkers to diagnose cardiac ischemia. More specifically, the biomarkers of the present invention can be used in diagnostic tests to determine, qualify, and/or assess ACI status, for example, to diagnose ACI, in an individual, subject or patient. In particular embodiments, cardiac ischemia status can include determining a patient's acute cardiac ischemia status or ACI status, for example, to diagnose ACI, in an individual, subject or patient. More specifically, the biomarkers to be detected in diagnosing cardiac ischemia (e.g., ACI) include, but are not limited to, SEQ ID NO:5, SEQ ID NO:6, SEQ ID NO:7, SEQ ID NO:8. SEQ ID NO:9, SEQ ID NO:10, SEQ ID NO:11 and SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28. Other biomarkers known in the relevant art may be used in combination with the biomarkers described herein.

A. Biomarker Panels

The biomarkers of the present invention can be used in diagnostic tests to assess, determine, and or qualify (used interchangeably herein) cardiac ischemia status in a patient. The phrase “cardiac ischemia status” includes any distinguishable manifestation of the condition, including not having cardiac ischemia. For example, cardiac ischemia status includes, without limitation, the presence or absence of cardiac ischemia in a patient, the risk of developing cardiac ischemia, the stage or severity of cardiac ischemia, the progress of cardiac ischemia (e.g., progress of cardiac ischemia over time) and the effectiveness or response to treatment of cardiac ischemia (e.g., clinical billow up and surveillance of cardiac ischemia after treatment). Based on this status, further procedures may be indicated, including additional diagnostic tests or therapeutic procedures or regimens.

The following Table shows the human serum albumin PTMs identified by the present inventors.

TABLE 1
Human Serum Albumin Post-Translationally Modified Peptides

| Peptide Sequence | Modification | Residues | Domain |
|---|---|---|---|
| ETYGEMADCCAK (SEQ ID NO: 5) | — | 105-116 | I |
| ETYGEMADCCAK (SEQ ID NO: 6) | Cysteinyl, C114 | 105-116 | I |
| ETYGEMADCCAK (SEQ ID NO: 7) | Phosphorylation, Y108 | 105-116 | I |
| TCVADESAENCDK (SEQ ID NO: 8) | — | 75-87 | I |
| TCVADESAENCDK (SEQ ID NO: 9) | Cysteinyl, C71 | 75-87 | I |
| TCVADESAENCDK (SEQ ID NO: 10) | Phosphorylation, S61 | 75-87 | I |
| ALVILIAFAQYLQQCPFEDHVK | — | 44-64 | I |
| (SEQ ID NO: 11) | | | |
| ALVLIAFAQYLQQCPFEDUVK | Cysteinyl, C57 | 44-64 | I |
| (SEQ ID NO: 12) | | | |
| LCIVATLR (SEQ ID NO: 13) | — | 97-104 | I |
| LCVIVTLR (SEQ ID NO: 14) | Cysteinyl, C98 | 97-104 | I |
| PEVDVMCTAFHDNEETFLK | — | 141-159 | I |
| (SEQ ID NO: 15) | | | |
| PEVDVMCTAFHDNEETFLK | Cysteinyl, C147 | 141-159 | I |
| (SEQ ID NO: 16) | | | |
| YLYEIAR (SEQ ID NO: 17) | — | 161-167 | I |
| YLYEIAR (SEQ ID NO: 18) | Phosphorylation, Y163 | 161-167 | I |
| AACLLPK (SEQ ID NO: 19) | — | 198-204 | I |
| AACLLPK (SEQ ID NO: 20) | Cysteinyl, C200 | 198-204 | I |
| YICENQDSISSK (SEQ ID NO: 21) | — | 286-297 | II |
| YICENQDSISSK (SEQ ID NO: 22) | Phosphorylation, S293 | 286-297 | II |
| YiCENQDSISSKI(SEQ ID NO: 23) | Phosphorylation, S295 | 286-297 | II |
| VPQVSTPTLVEVSR (SEQ ID NO: 24) | — | 438-451 | III |
| VPQVSTPTIVEVSR (SEQ ID NO: 25) | Phosphorylation, T445 | 438-451 | III |
| VPQVSTPTINEVSR (SEQ ID NO: 26) | Phosphorylation, T443 | 438-451 | III |
| VPQVSTPTLVEVSR (SEQ ID NO: 27) | Phosphorylation, S442 | 438-451 | III |
| VPQVSTPTLVEVSR (SEQ ID NO: 28) | Double | 438-451 | III |
| | Phosphorylation, S442, | | |
| | T443 | | |
| GDFQFNISR (SEQ ID NO: 29) | External Standard | n/a | n/a |
| TYETTLEK (SEQ ID NO: 30) | Internal Standard | 375-457 | II |

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the biomarker panels of the present invention may show a statistical difference in different ACI statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

The biomarkers are differentially present in UI (NC or non-cardiac ischemia) and cardiac ischemia, and, therefore, are useful in aiding in the determination of cardiac ischemia status. In certain embodiments, the biomarkers are measured in a patient sample using the methods described herein and compared, for example, to predefined biomarker levels and correlated to cardiac ischemia status. In particular embodiments, the measurement(s) may then be compared with a relevant diagnostic amount(s), cut-off(s), or multivariate model scores that distinguish a positive cardiac ischemia status from a negative cardiac ischemia status. The diagnostic amount(s) represents a measured amount of a biomarker(s) above which or below which a patient is classified as having a particular cardiac ischemia status. For example, if the biomarker(s) is/are up-regulated compared to normal during cardiac ischemia, then a measured amount(s) above the diagnostic cutoff(s) provides a diagnosis of cardiac ischemia. Alternatively, if the biomarker(s) is/are down-regulated during cardiac ischemia, then a measured amount(s) at or below the diagnostic cutoff(s) provides a diagnosis of non-cardiac ischemia. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarkers in a statistically significant number of samples from patients with the different cardiac ischemia statuses, and drawing the cut-off to suit the desired levels of specificity and sensitivity.

Indeed, as the skilled artisan will appreciate there are many ways to use the measurements of two or more biomarkers in order to improve the diagnostic question under investigation. In a quite simple, but nonetheless often effective approach, a positive result is assumed if a sample is positive for at least one of the markers investigated.

Furthermore, in certain embodiments, the values measured for markers of a biomarker panel are mathematically combined and the combined value is correlated to the underlying diagnostic question. Biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g. SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating a biomarker combination of the present invention, e.g. to diagnose cardiac ischemia, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D). G., Pattern Classification, Wiley Interscience, 2nd Edition (2001).

B. Determining Risk of Developing Cardiac Ischemia

In a specific embodiment, the present invention provides methods for determining the risk of developing cardiac ischemia in a patient. Biomarker percentages, amounts or patterns are characteristic of various risk states, e.g., high, medium or low. The risk of developing cardiac ischemia is determined by measuring the relevant biomarkers and then either submitting them to a classification algorithm or comparing them with a reference amount. i.e., a predefined level or pattern of biomarkers that is associated with the particular risk level.

C. Determining Cardiac Ischemia Severity

In another embodiment, the present invention provides methods for determining the severity of cardiac ischemia in a patient. Each grade or stage of cardiac ischemia likely has a characteristic level of a biomarker or relative levels of a set of biomarkers (a pattern). The severity of cardiac ischemia is determined by measuring the relevant biomarkers and then either submitting them to a classification algorithm or comparing them with a reference amount, i.e., a predefined level or pattern of biomarkers that is associated with the particular stage.

D). Determining Cardiac Ischemia Prognosis

In one embodiment, the present invention provides methods for determining the course of cardiac ischemia in a patient. Cardiac ischemia course refers to changes in cardiac ischemia status over time, including cardiac ischemia progression (worsening) and cardiac ischemia regression (improvement). Over time, the amount or relative amount (e.g., the pattern) of the biomarkers changes. For example, biomarker “X” may be increased with cardiac ischemia, while biomarker “Y” may be decreased with cardiac ischemia. Therefore, the trend of these biomarkers, either increased or decreased over time toward cardiac ischemia or non-cardiac ischemia indicates the course of the condition. Accordingly, this method involves measuring the level of one or more biomarkers in a patient at least two different time points, e.g., a first time and a second time, and comparing the change, if any. The course of cardiac ischemia is determined based on these comparisons.

E. Patient Management

In certain embodiments of the methods of qualifying cardiac ischemia status, the methods further comprise managing patient treatment based on the status. Such management includes the actions of the physician or clinician subsequent to determining cardiac ischemia status. For example, if a physician makes a diagnosis of cardiac ischemia, then a certain regime of monitoring would follow. An assessment of the course of cardiac ischemia using the methods of the present invention may then require a certain cardiac ischemia therapy regimen. Alternatively, a diagnosis of non-cardiac ischemia might be followed with further testing to determine a specific disease that the patient might be suffering from. Also, further tests may be called for if the diagnostic test gives an inconclusive result on cardiac ischemia status.

F. Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of one or more of the biomarkers of the present invention may change toward a non-cardiac ischemia profile. Therefore, one can follow the course of one or more biomarkers in the patient during the course of treatment. Accordingly, this method involves measuring one or more biomarkers in a patient receiving drug therapy, and correlating the biomarker levels with the cardiac ischemia status of the patient (e.g., by comparison to predefined levels of the biomarkers that correspond to different cardiac ischemia statuses). One embodiment of this method involves determining the levels of one or more biomarkers for at least two different time points during a course of drug therapy, e.g., a first time and a second time, and comparing the change in levels of the biomarkers, if any. For example, the levels of one or more biomarkers can be measured before and after drug administration or at two different time points during drug administration. The effect of therapy is determined based on these comparisons. If a treatment is effective, then the one or more biomarkers will trend toward normal, while if treatment is ineffective, the one or more biomarkers will trend toward cardiac ischemia indications.

G. Generation of Classification Algorithms for Qualifying Cardiac Ischemia Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g. Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows® or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

The learning algorithms described above are useful both for developing classification algorithms for the biomarkers already discovered, and for finding new biomarker biomarkers. The classification algorithms, in turn, form the base for diagnostic tests by providing diagnostic values (e.g., cut-off points) for biomarkers used singly or in combination.

H. Kits for the Detection of Cardiac Ischemia Biomarkers

In another aspect, the present invention provides kits for qualifying cardiac ischemia status, which kits are used to detect the biomarkers described herein. In a specific embodiment, the kit is provided as an ELISA kit comprising antibodies to the biomarkers of the present invention including, but not limited to, SEQ ID NO:5, SEQ ID NO:6, SEQ ID NO:7, SEQ ID NO:8, SEQ ID NO:9, SEQ ID NO:10, SEQ ID NO:11 and SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28. In such embodiments, the antibodies specifically bind to the modified or unmodified forms of human serum albumin as indicated in the relevant sequence identification numbers.

The ELISA kit may comprise a solid support, such as a chip, microtiter plate (e.g., a 96-well plate), bead, or resin having biomarker capture reagents attached thereon. The kit may further comprise a means for detecting the biomarkers, such as antibodies, and a secondary antibody-signal complex such as horseradish peroxidase (HRP)-conjugated goat anti-rabbit IgG antibody and tetramethyl benzidine (TMB) as a substrate for HRP.

The kit for qualifying cardiac ischemia status may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

In certain embodiments, a patient can be diagnosed by adding blood or blood serum from the patient to the kit and detecting the relevant biomarkers conjugated with antibodies, specifically, by a method which comprises the steps of: (i) collecting blood or blood serum from the patient; (ii) separating blood serum from the patient's blood; (iii) adding the blood serum from patient to a diagnostic kit; and, (iv) detecting the biomarkers conjugated with antibodies. In this method, the antibodies are brought into contact with the patient's blood. If the biomarkers are present in the sample, the antibodies will bind to the sample, or a portion thereof. In other kit and diagnostic embodiments, blood or blood serum need not be collected from the patient (i.e., it is already collected). Moreover, in other embodiments, the sample may comprise a tissue sample or a clinical sample.

The kit can also comprise a washing solution or instructions for making a washing solution, in which the combination of the capture reagents and the washing solution allows capture of the biomarkers on the solid support for subsequent detection by, e.g., antibodies or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected, etc. In yet another embodiment, the kit can comprise one or more containers with biomarker samples, to be used as standard(s) for calibration.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example I

### Characterizing Human Serum Albumin Biomarker Peptides During Acute Cardiac Ischemia Using Selected Reaction Monitoring Mass Spectrometry (SRM-MS)

During a heart attack, the cellular necrosis that characterizes an acute myocardial infarction (AMI) is first preceded by a period of reversible oxygen deprivation called acute cardiac ischemia (ACI). Diagnosing an AMI in the current clinical setting is fairly easy. A host of diagnostic biomarkers have been approved by the United States Food and Drug Administration for assaying myocardial necrosis; administering the most popular (that for cardiac troponin) has been so successful during patient evaluation and triage that the test has become a medical requirement. Unfortunately, no comparable ischemic diagnostic marker has been identified and ACI patient assessment remains a significant clinical challenge.

The present inventors have developed an eight peptide multiplexed selected reaction monitoring (SRM) assay using an LC-based sample preparation workstation (Perfinity Workstation) coupled with mass spectrometry to facilitate selective quantitation of five first domain human serum albumin post translational modifications (PTMs) in plasma. The limit of detection for the assay was determined at one femtomole per μL. Using the developed SRM method, cardiac sinus plasma samples were analyzed from eight clinical subjects taken at six time points immediately before, during, and following aortic valve replacement surgery. Computational analysis was performed following SRM screening using Cardiac Troponin I measurements to assess cardiac injury. Assay results were then used to characterize the post-translational modification (PTM) profile of human serum albumin's amino-terminal. SRM data suggested different levels of agrin, plasma kallikrein and putative myosin-XVB in SPMS patients as compared to healthy controls. Overall, an efficient, high throughput platform to characterize PTM-based biomarkers in plasma has been established which could easily be adapted for other proteins of interest related to cardiac disease.

A rapid, highly specific and sensitive multiplex SRM-MS assay was established to characterize amino-terminal PTMs of human albumin during mild ischemia and infarction. Integrating the Perfinity Workstation into the SRM-based platform dramatically improved both the reproducibility and throughput of the assay. Three proteins were found to be expressed significantly differently in SPMS patients as compared to health controls. These findings help qualify ischemic modified albumin as it relates to cardiac ischemia, enhancing the current understanding of ischemic heart disease pathology.

### Materials and Methods

Clinical Samples.

Both serum and plasma samples were used in this study. Serum used during initial method development efforts was purchased as a single 10 mL pool from Bioreclamation (Bioreclamation, LLC). The sera was collected at a FDA licensed and inspected Donor Center. Ten healthy, consenting, paid males between 20-60 years of age who had completed a written health screening prior to blood collection and had no history of heart disease or diabetes were selected as donors. Following whole blood donation, blood samples were allowed to clot, spun into serum. 0.21 μm filtered to eliminate contaminants, and then pooled. Post-collection, all commercially purchased sera viral tested negative for: HBsAg, HIV 1/2 Ab, HIV-1 RNA, HCV Ab, HCV RNA, & STS. For the SRM patient screen, cardiac sinus plasma samples from a total of eight clinical subjects were obtained at six time points before, during, and after aortic valve replacement surgery. For each patient, the first samples were acquired immediate prior (T0) and 5 minutes following (T1) removal of the individual from cardiopulmonary bypass during surgery. The last sample (T9) was drawn thirty minutes after surgery completion. The operative technique was similar in all patients, with an average aortic cross clamp time of 55.6 minutes.

Sample Preparation.

To minimize the potential for ex-vivo enzymatic-based cleavage or sample degradation as a result of sample handling, all samples were processed and prepared using the standard operating procedure detailed here. Blood collections followed standard operating procedures including: time to centrifugation, centrifugation speed, storage, and shipment conditions for all processed samples. Upon purchase, the 10 mL pooled sample was packed in dry ice and shipped overnight in an insulated styroform container. Upon receipt, the sample was divided into individual 30 μl aliquots and then cryoperserved at −80° C. The plasma samples were packed, shipped, and stored using a similar protocol however, the plasma samples were aliquoted into individual 12 μl aliquots before cryopreservation.

External Standard Sample Preparation.

A stock of 1 pmol/μL of intact E. coli beta-galactosidase (Sigma Aldrich) was prepared in deionized water to be used as an external standard spiked into all analysed sera and plasma samples.

Optimizing Front-End Separation Conditions.

Prior to the eight clinical patients screen, all front-end conditions were tested and optimized using control sera. The final optimized conditions are detailed below. Ultimately, using the configured platform, trypsin digestion, desalting, and LC-SRM was completed in 35 minutes for each injected sample.

A. Pre-Injection Sample Preparation.

A 50-fold dilution of all analysed sera and plasma samples was constructed using 480 μL of 0.1% Rapigest (Waters, Milford, Mass.) resuspended in 100 mM ammonium bicarbonate, 10 μL of the 1 pmol/μL stock of the external protein standard, intact E. coli beta-galactosidase, and 10 μL of undiluted sera or plasma for a final total volume of 500 μL for each processed sample. Each sample aliquot was placed into the Shimadzu Sil-20 AC Autosampler (Shimadzu, Columbia, Md.) of the Perfinity Work-station (Perfinity Biosciences, Inc.), maintained at a constant temperature of 6° C. Individual plasma samples used during cohort screening were prepared in parallel to minimize experimental error during the sample preparation. Plate position and order within sample queue was determined via random number generator for all patient screen experiments. All reagents used during sample preparation were purchased from Sigma Aldrich if not mentioned specifically.

B. Sample Injection.

Fifty microliters of each diluted sera or plasma sample was injected onto the Perfinity Workstation (Perfinity Biosciences, Inc.) per individual analysis.

C. On-Column Trypsin Digestion and Desalt.

To preserve all endogenous human serum albumin cysteinyl modifications, no protein disulfide reduction procedure was performed prior to trypsinization. Samples were incubated in the Shimadzu CTO-20AC Column Oven Chamber of the Perfinity Workstation (Perfinity Biosciences, Inc.) at 50° C. for the entire sample preparation process. Digestion within the immobilized enzyme reaction chamber proceeded for six minutes. Digested sample was then automatically injected onto a Perfinity desalt column and on-line column based sample desalting was performed. All buffers used during sample preparation were commercially prepared and obtained by Perfinity Biosciences, Inc.

D. Optimizing LC Separations.

A Halo c18 column (100×2.1 mm, 2.7 μm) (Advanced Materials Technology, Wilmington, Del.) was selected for use during LC separation. UV-vis HPLC Chromatograms were acquired using the Shimadzu SPD-20A UV-V is detector incorporated into the Perfinity Workstation's (UV-vis detector operated at a wavelength of 214 nm). A 20 minute RP-HPLC separation was performed for all sample separations whereby peptides were eluted over a linear gradient with initial concentration of solvent B at 5% B and final concentration of solvent B at 42% B. Solvent A consisted of 0.1% formic acid in water and solvent B consisted of 90% acetonitrile, 10% water and 0.1% formic acid although both solvents A and 1 were prepared by and obtained from Perfinity Biosciences, Inc. The flow rate of the Perfinity Workstation binary pump was set to 500 μL/min. All LC runs were immediately followed by two five minute high-flow column washes (5 minute high organic linear ramp-downs, performed at a flow rate of 4 ml/min).

LOD of LC-MS/MS Method.

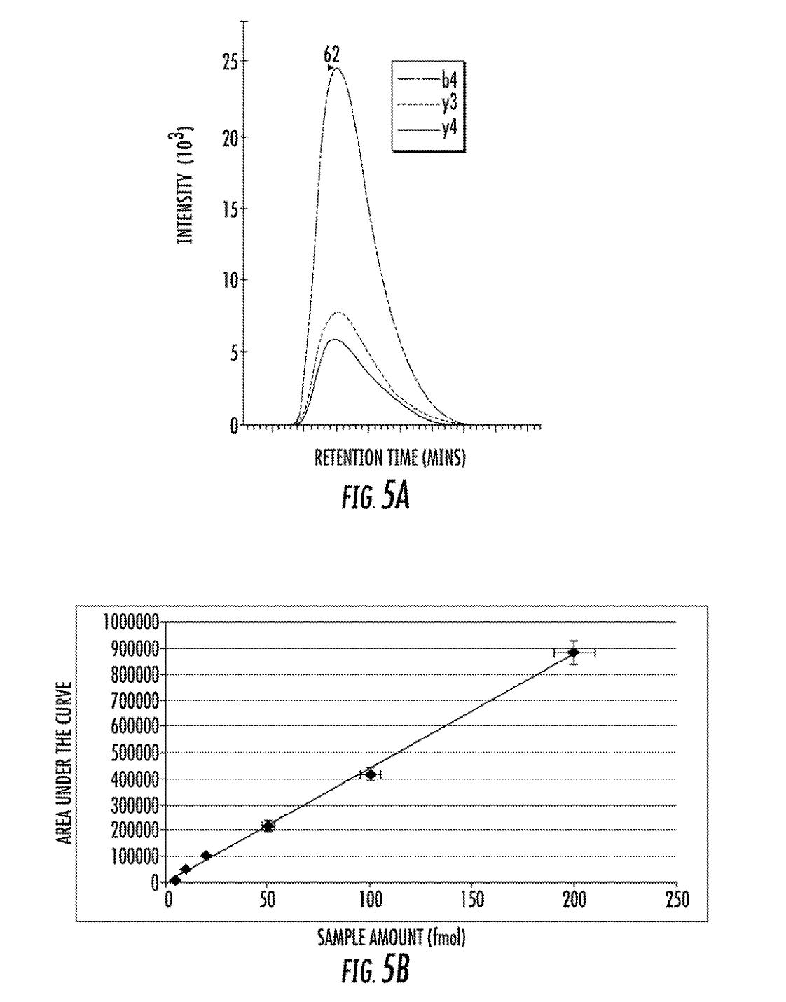

Intact E. coli beta-galactosidase was chosen as a model protein to establish the LOD of our multiplex SRM LC-MS/MS method. The protein was diluted serially in sera with concentrations ranging from 1 fmol/μL to 200 fmol/μL. Using a sera sample with 100 fmol/μL of E. coli beta-galactosidase, the following was experimentally determined: the signature tryptic peptide GDFQFNISR (SEQ ID NO:29) was selected as the surrogate peptide for the beta-galactosidase protein, the three most abundant detected transitions of peptide GDFQFNISR (SEQ ID NO:29), y ion y2, y4, and y5 were selected for detection, and the most abundant, y5, was selected to generate the standard curve. The series of beta-galactosidase protein dilutions were then subject to LC-MS/MS analysis in the Thermo TSQ Vantage mass spectrometer with the Q1 mass filter targeting the parent ion of GDFQFNISR (SEQ ID NO:29) and Q3 mass filter targeting detection of the y2, y4, and y5 product ions. Ions were introduced into the mass spectrometer via an H-ESI II probe outfitted with a 32 gauge needle (Thermo Fisher Scientific, Waltham, Mass., USA). Instrument operating parameters used during acquisition were as follows: capillary temperature of 380° C., vaporizing temperature of 400° C., sheath gas pressure was set at 60, auxiliary gas pressure was set at 20, and the spray voltage was set to 4,000 V. Instrument settings in the TSQ Vantage operating software (Xcaliber v 2.1, Thermo Fisher Scientific) were as follows: positive scanning mode, chrom filters enabled and set at 70, collision gas set at 1.5 mTorr, Q1 peak width (FWHM) of 0.7. Q3 peak width of (FWHM) 0.7, and cycle times of 0.1 seconds. Following data acquisition, peak areas under the curve were calculated using the open source proteomic software platform Skyline [40] (Skyline v. 1.2, MacCoss Lab, Seattle, Wash.) and a linear standard curve (y=4390.1x−932.73 R²=0.9982) was generated using Microsoft Excel (FIG. 5B).

LC-MS/MS Method Development and Optimization.

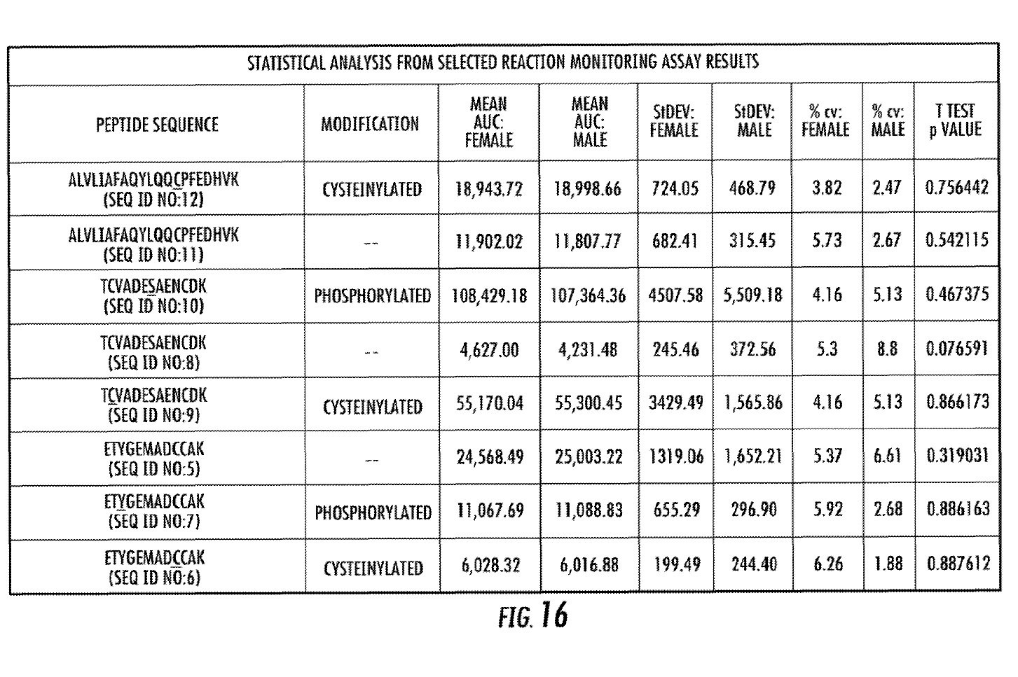

Using the instrument platform described above, we designed a multiplexed peptide based SRM assay using all sample preparation and mass spectrometric settings mentioned in the previous method sections. Our assay allows for the simultaneous relative quantitation of eight tryptic peptides originating from the N-terminus of human serum albumin in either plasma or sera. The FASTA files for both human serum albumin and E. coli beta-galactosidase were downloaded from Uniprot (www.uniprot.org) individually and imported into the Skyline software program. Instrument methods were exported directly from the Skyline program into an Xcaliber “EZ SRM” method template with the same settings as used during data acquisition for our LOD determination experiments. To design the SRM assay, an iterative method development strategy was adopted, eliminating poor performing transitions from the assay in a targeted method refinement cycle. Raw data was inspected in the Skyline software platform and the peak areas of each detected transition were normalized to that of a beta-galactosidase trypsinized peptide GDFQFNISR (SEQ ID NO:29) and an internal albumin peptide TYETTLEK (SEQ ID NO:30) to minimize run-to-run variability as a function of instrument performance. Starting with an initial 358 transitions for the ten selected peptides, multiple rounds of iterative method refinement were completed to generate the final SRM assay. The final SRM assay is a scheduled method consisting of 39 transitions with a scheduling window between two and four minutes. Reproducibility was determined via Precision Calculation of the patient screen and % cv values for each measured peptide are displayed in FIG. 16.

Data Analysis.

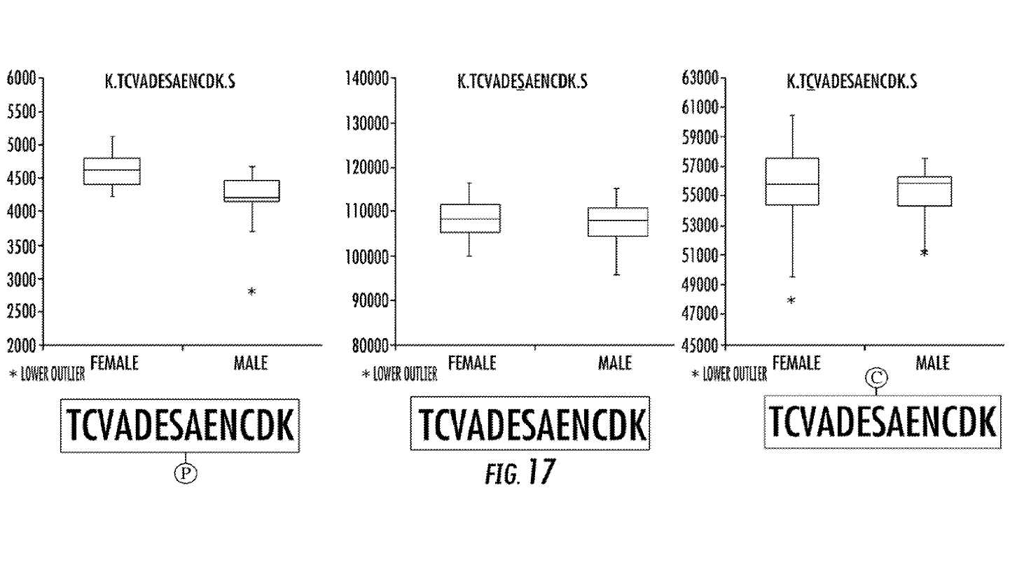

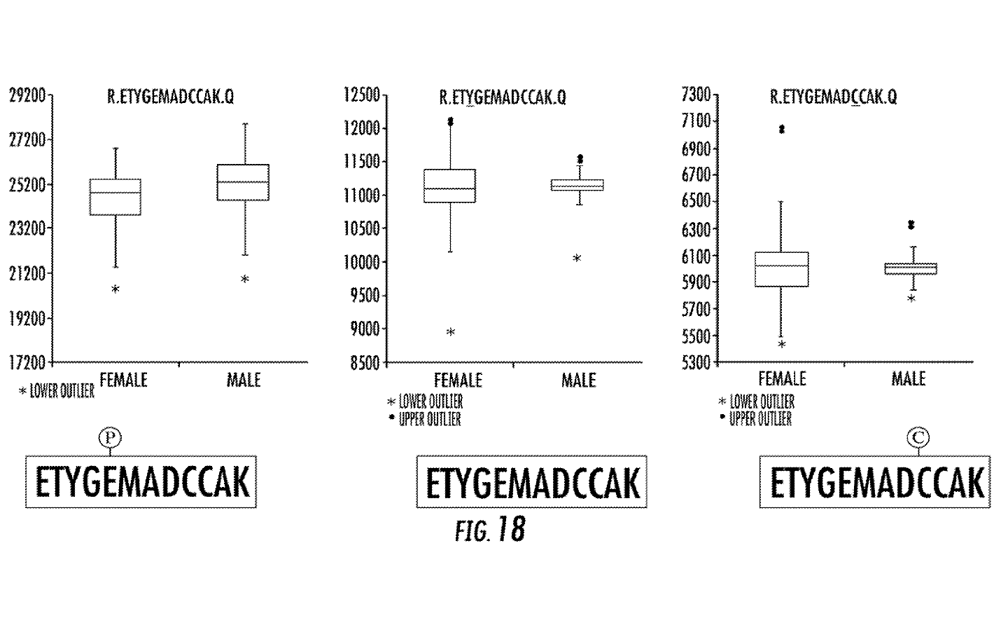

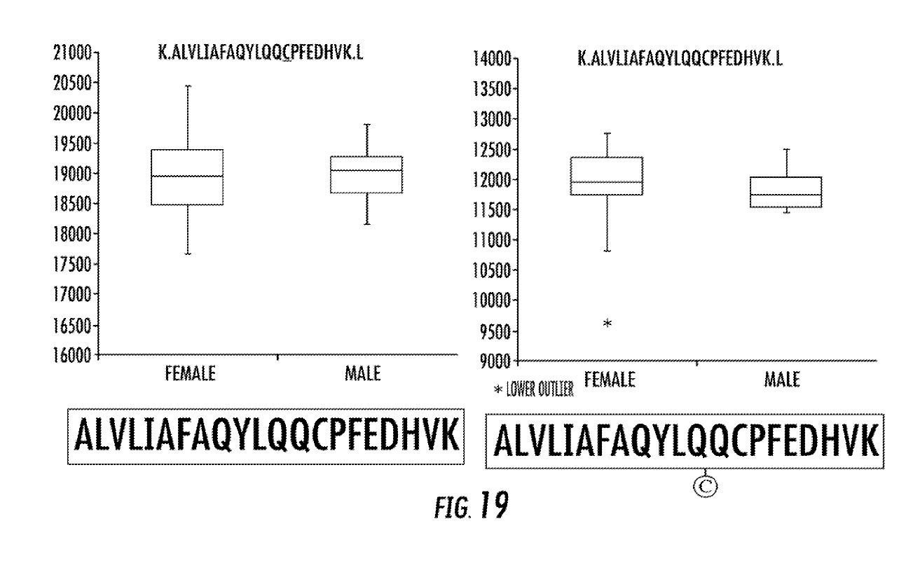

Differences in peptide expression level between the tested male and female control subjects were constructed into box plots and analyzed by the t test. For all t tests, p values <0.05 were considered significant. Results stemming from statistical analysis are displayed in FIG. 16, box plots for each of the eight peptides analyzed are in FIGS. 17-19.

### Results

Albumin Protein Post-Translational Modifications.

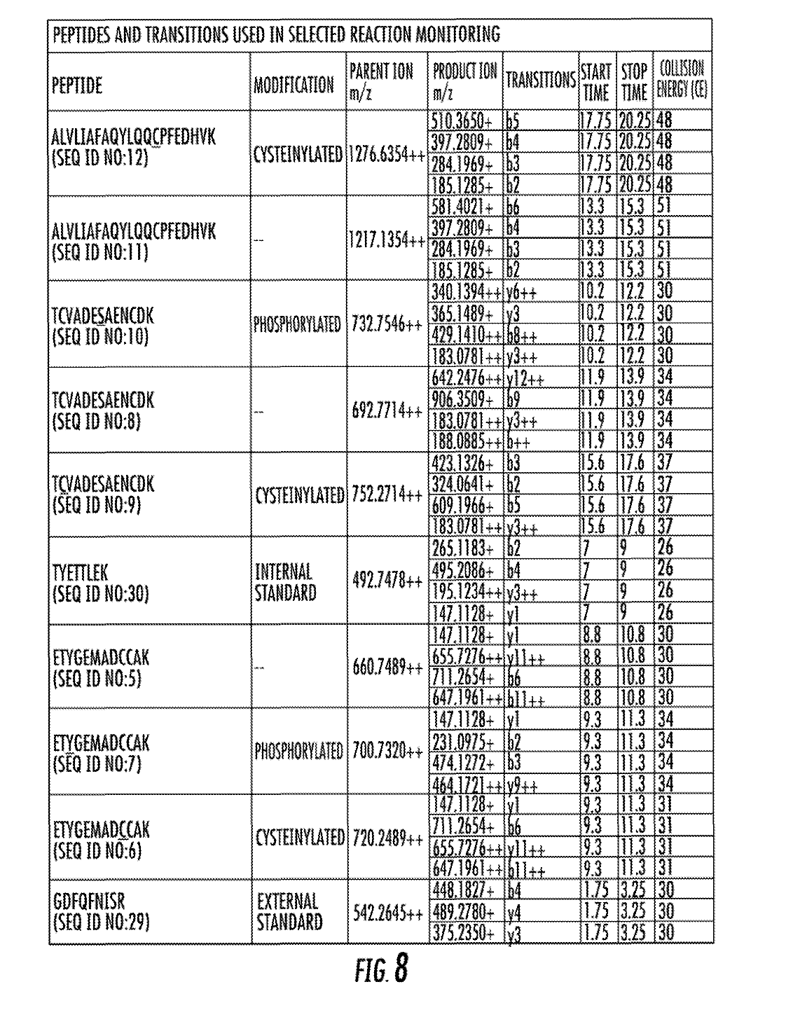

The list of human serum albumin peptide sequences and PTMs selected for this study is listed in FIG. 8. In this embodiment, only PTMs in close proximity to albumin's amino metal binding site directly or are at the site of Albumin's free cysteine residue, Cys34, were chosen. As such, the three cysteinyl modifications, single serine phosphorylation, and tyrosine phosphorylation included are all localized to the first domain of human serum albumin. Of the five PTMs selected, only three had been identified as human serum albumin PTMs previously. See Han et al., 8 PROTOMICS 1346-61 (2008); Rikova et al. 131 CELL 1190-1203 (2007); Kleinova et al., 19(20) RAPID COMMUN. MASS SPECTROM. 2965-73 (2005); and Saber et al., 42 COLLECT. CZECH. CHEM. COMMUN. 564-79 (1977). The two novel cysteinyl modifications were identified by the present inventors directly during initial protein characterization experiments via bottom-up targeted mass spectrometry with a high resolution LTQ-OrbitrapXL. Prior to SRM method development, the existence of all five human serum albumin PTMs were confirmed with this high resolution mass spectrometry platform using pooled sera obtained commercially (Bioreclamation, LLC). MS2 spectra from the validation efforts has been included in FIGS. 17-19. A de novo sequencing approach was used to confirm peptide sequence.

Sampling.

To ensure that SRM results of the tested patient samples reflected only in-vivo human serum albumin micro-heterogeneities and not artificially-induced changes, a rigorous sample preparation protocol was devised and followed for every patient sample analysed. All plasma was stored at −80° C., thawed only twice, and exposure to room temperature during sample preparation for limited to five minutes. All samples were blinded prior to any experimentation. During screening, samples were analysed in batches whereby all six samples for a single patient were analysed as a block. The SRM sample queue for each block of samples was determined using a random number generator; the ordering of samples within a sample block was also randomized. SRM analysis of each sample block was performed in duplicate. The list of human serum albumin peptide sequences monitored, the PTMs monitored, the specific fragmentation transitions, the optimized collision energy (CE), and the start/stop times in the scheduled SRM experiment are listed in FIG. 8. A signature peptide from E. coli beta-galactosidase (Sigma Aldrich, St. Louis, Mo.) was used as an external standard for determination of detection limits. An additional signature human serum albumin peptide was used to direct statistical normalization during quantitation.

Trypsin Digestion Efficiency.

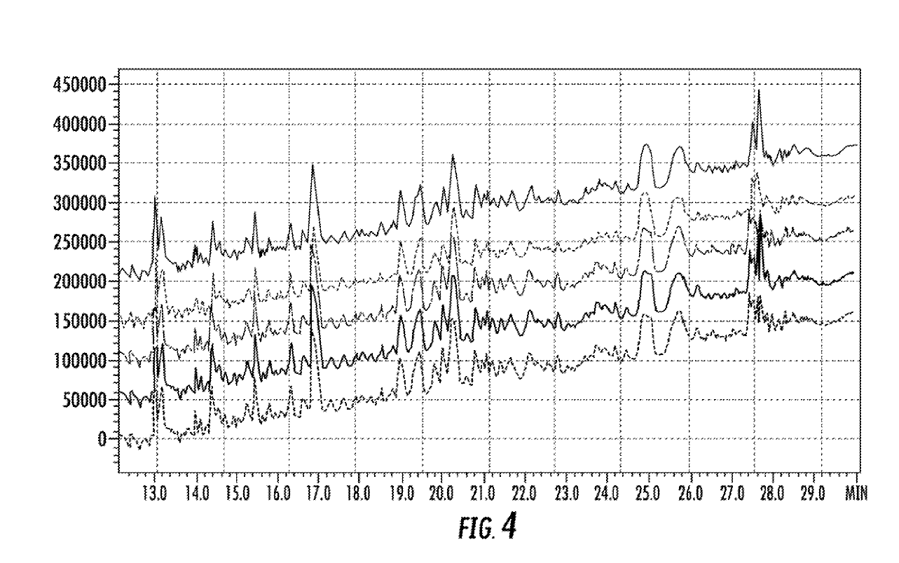

The first experiments performed with the Perfinity Workstation (Perfinity Biosciences, Inc) optimized on-column sample trypsinization using pooled sera obtained commercially (Bioreclamation, LLC). Using the pooled sera sample (Bioreclamation, LLC) as a standard test matrix, UV-vis based detection of the tryptic peptide map (a c18-based linear RP-HPLC separation of the tryptic digest) was used to measure experimental digestion efficiency, reproducibility, and percentage sample carryover. To obtain the sample replicate comparison shown in FIG. 4, digestion using the pooled sera sample was repeated five times. From this experiment, the efficiency of the digestion process was determined to be approximately 90%. This measurement was obtained by calculating the ratio between the areas under the curves of chromatographic peaks eluted during the biological gradient (5%-75%) and the areas under the curves of undigested peptide peaks eluted at high organic (75-95%). Percentage carryover between sample replicates was calculated by comparing area under the curve measurements of chromatographic peaks detected during analysis using 50 μL of diluted sera to area under the curve measurements of chromatographic peaks detected with a null injection following analysis of a sera sample and end-of-run column washes described above. By incorporating two back-to-back column washes between samples, sample contamination from carryover from previous sample injections was almost negligible (data not shown).

Limit of Detection Retention Time and Peak Area Reproducibility.

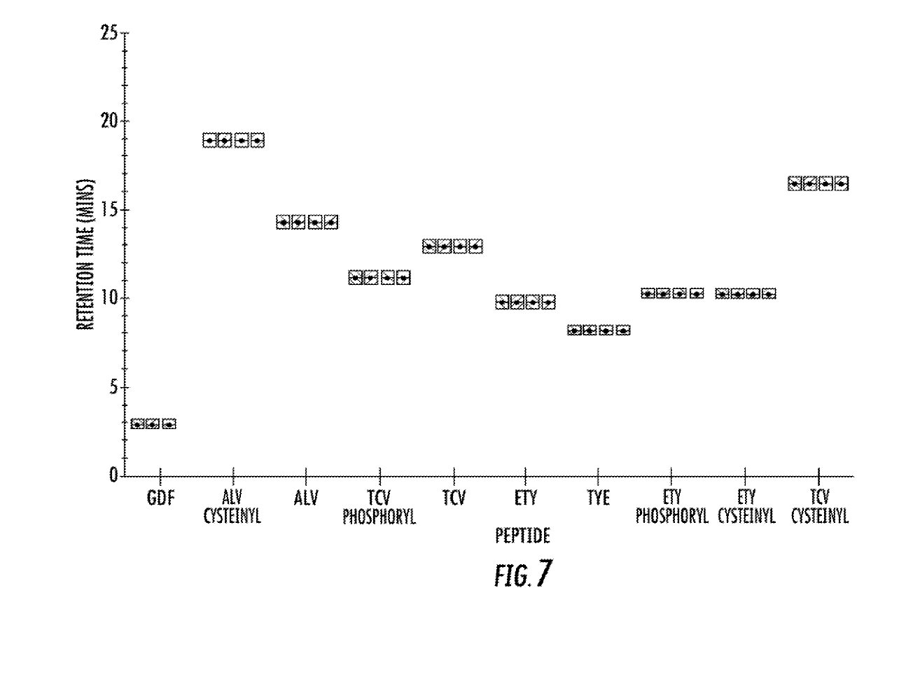

Using the sample preparation conditions determined initially, intact E. coli beta-galctosidase (Sigma Aldrich) was serially diluted and added to the pooled control subject sera to determine matrix affects (Bioreclamation, LLC). SRM analysis was performed to detect the three most abundant transitions of a signature tryptic beta-galactosidase peptide (generated during on-line sample preparation with the Perfinity Workstation). As shown in FIG. 5B, using seven serial dilutions, the limit of detection of the assay platform was determined as one femtomole. These measurements monitored the peptide GDFQFNISR MH₂⁺² ion transitions to the y₃, y₄ and b₄ fragment ions at a retention time of 2.9 minutes as shown in FIG. 5A. Retention time reproducibility for the eight diagnostic human serum albumin peptides, the signature internal standard, and the external standard included in the ACI assay we developed were obtained from three technical replicates performed using the pooled control subject test sample and are shown in FIG. 7.

### Discussion

A diagnostic ischemic biomarker may facilitate early detection of ACI, increasing both the total number of patients properly diagnosed and the rate at which this diagnosis is achieved. No “gold standard” currently exists for assessing myocardial ischemia in clinical subjects. As such, using clinical cohorts to assess the diagnostic power of a candidate biomarker specific to ischemia can mire validation efforts. In the present study. IMA was defined by a limited PTM profile for human serum albumin. The diagnostic power of the PTM biomarker panel was determined in an induced ischemic model whereby no ambiguity existed regarding the patient's medical condition. The surgical model simulated a patient's progression (from normal physiology to ischemia to infarction) during ischemic heart disease but in a way that was both controlled and measurable. The six acquired time points were correlated to disease state (non-ischemic, mild ischemic, mild infarction) by the respective amount of cardiac tissue damage detected in each sample via cardiac troponin I measurements. This longitudinal sampling approach facilitated validating IMA using a comparative SRM assay. Clinical biomarkers must reflect biological changes that can be directly attributed to the pathology of a specific disease. The use of a three state disease model ensured that the SRM results could be correlated to ischemia specifically and differentiated from a control baseline state or infarction.

The work here was performed using a relatively small cohort since the intent was to qualify the diagnostic value of using PTMs to distinguish IMA from unaltered human albumin. To determine if the eight human serum albumin peptides qualified here have true clinical value for cardiac ischemic, large scale validation experiments using cohorts of clinical samples will be required.

The present study does prove the feasibility of incorporating automated sample preparation into the SRM workflow. Using the Perfinity Workstation dramatically reduces the time associated with proteomic sample preparation (from 18 hour digestion to six minute digestion) while increasing the reproducibility of the process (less than 10% cvs for all detected peptides). Using SRM-mass spectrometry with the Perfinity Workstation, a highly reproducible 35 minute assay has been established and up to 25 samples were able to be screened a day using the platform. Additionally, while the SRM assay targeted human serum albumin, it is important to note that one of the key advantages of the dual instrument platform we employed is its flexibility in target selection. The current instrument workflow could easily be transformed to validate other protein biomarkers or other modifications of interest.

Both ELISA and mass spectrometric platforms have the sensitivity necessary to detect a highly abundant plasma protein biomarker like human serum albumin. However, in part because of the ease in which high abundant proteins can be detected, the diagnostic value of these proteins remains largely a mystery. Proteomic-based biomarker development efforts almost exclusively ignore high abundant plasma proteins during discovery and validation efforts. Rather, proteins “leaked” into plasma from diseased tissues are prioritized during biomarker candidate selection. The purported value of tissue leakage products as biomarkers stems from the assumption that tissue at the primary localized site of disease holds the greatest reservoir of disease indicators. Generally speaking, tissue leakage products are present in plasma at 10⁴-10⁶-fold lower abundance than the five most abundant plasma proteins. To facilitate detection of low abundant tissue leakage products with the current dynamic range limitations of commercial mass spectrometers, depleting plasma samples of the most abundant protein fraction has become routine during biomarker development. A better understanding of the biology of plasma biomarkers would help develop more meaningful criteria for prioritizing potential candidates. During blood circulation, several research groups have proven that components of the blood proteome adopt molecular cues from local tissue environments as a function of disease perturbed or disease activated cellular networks. If diseased tissues are releasing metabolites or free radical ions into the blood as a function of localized damage, once in the blood supply, these species should necessarily interact with higher abundant proteins with higher frequency than with lower abundant plasma proteins. Thus, from the point of injury to the time of sampling, high abundant plasma proteins should have the greatest potential to change in response to the changing plasma metabalome. While validating high abundant plasma markers with disease should not preclude current efforts correlating disease with tissue leakage products, high abundant plasma proteins should be assessed for their value as clinical biomarkers.

### CONCLUSION

Overall, the present inventors have established a straightforward mass spectrometry-based platform for characterizing PTMs and verifying plasma peptide biomarkers that can be easily adapted to study a broad range of proteins and modifications, especially those of interest to cardiac disease. The statistical analysis suggests that amine-terminal albumin PTMs may be responsive to ischemia and the developed PTM profile may have value as a clinical ACI diagnostic.

### Example 2

### Using Automated Sample Preparation to Increase the Utility of Selected Reaction Monitoring Mass Spectrometry (SRM-MS) in Emergency Department Diagnostics

Using a LC-system (Perfinity Workstation) that automates front-end sample preparation coupled with mass spectrometry, the present inventors have established a highly specific and sensitive multiplex selected reaction monitoring (SRM) assay compatible with ACI diagnostic requirements. The 35 minute SRM assay has facilitated the simultaneous detection of eight N-terminal domain human serum albumin (HSA) peptides. To further test the robustness and reproducibility of the SRM-assay platform, 30 sera samples from 15 male and 15 female control subjects were analyzed in parallel. The screen was then followed by computational analysis for quantitation. The SRM data suggest that significant gains in reproducibility and speed can be achieved if front-end sample preparation is moved from a manual multi-step benchtop process to an automated column-mediated format. By modifying sample preparation, the present inventors have established an efficient platform to verify cardiac ischemic peptide biomarkers in sera which can be easily adapted to other proteins of interest related to cardiac disease.

A rapid, highly specific and sensitive multiplex SRM-MS assay was established for verifying sera peptide biomarkers. The eight peptides selected for inclusion were not differentially expressed as a function of the gender of the subjects that were screened. Automating the sample preparation required for analysis by SRM dramatically improved both the reproducibility and throughput of the screening technique. Ultimately, the Perfinity Workstation coupled with mass spectrometry provides a novel, robust, and rapid assay platform well-suited for an emergency department setting.

### Materials and Methods

Clinical Samples.

Sera samples involved in the study were obtained commercially (Bioreclamation, LLC) from an FDA licensed and inspected Donor Center. All blood samples were obtained from healthy, consenting, paid donors between 20-60 years of age who had completed a written health screening prior to blood collection and had no history of heart disease or diabetes. Two sets of samples were obtained—a single 10 mL pooled sera sample collected from ten healthy male donors and a cohort consisting of three 0.1 mL aliquots of sera from 15 male subjects and 15 female subjects (90 samples in total). Following whole blood donation, blood samples were allowed to clot, spun into serum, 0.2 μm filtered to eliminate contaminants, sequentially coded and aliquoted according to specifications. Post-collection, all sera viral tested negative for: HBsAg, HIV 1/2 Ab, HIV-1 RNA, HCV Ab, HCV RNA. & STS.

Sera Sample Preparation.

To minimize the potential for ex-vivo enzymatic-based cleavage or sample degradation as a result of sample handling, all sera samples were processed and prepared using the standard operating procedure detailed here. Blood collections followed standard operating procedures including: time to centrifugation, centrifugation speed, storage, and shipment conditions for all processed samples. Upon purchase, samples were packed in dry ice and shipped overnight in insulated styroform containers. The 10 mL pooled sera sample (Bioreclamation, LLC) was divided into individual 30 μl aliquots and then cryoperserved at −80° C. The 90 0.1 mL sera samples (Bioreclamation, LLC) were not further aliquoted, but were immediately cryopreserved at −80° C. upon receipt. Each 0.1 mL aliquot of sera was thawed only immediately before analysis and any material not used was discarded rather than re-frozen.

Subjects.

The 10 mL pooled sera sample was used during initial method development. The ten participants used to construct the pooled sample were different from the male subjects included in the 30 person cohort. To minimize the influence age and/or gender may have had the 30 person SRM-screen, samples were paired, constructing male female sample pairs of similarly aged individuals. Sample pairs were then analyzed in duplicate.

External Standard Sample Preparation.

The method is the same as described above in Example 1.

Optimizing Front-End Separation Conditions.

The methods, specifically, pre-injection sample preparation, sample injection, on-column trypsin digestion and desalt, and optimization of LC separation, were the same as described above in Example 1.

LC-MS/MS Method Development and Optimization.

Using the instrument platform described above, a multiplexed peptide based SRM assay was designed using all sample preparation and mass spectrometric settings mentioned in the previous method sections. In one embodiment, the assay of the present invention allows for the simultaneous relative quantitation of eight tryptic peptides originating from the N-terminus of human serum albumin. The FASTA files for both human serum albumin and E. coli beta-galactosidase were downloaded from Uniprot individually and imported into the Skyline software program. Instrument methods were exported directly from the Skyline program into an Xcaliber “EZ SRM” method template with the same settings as used during data acquisition for the LOD determination experiments. To design the SRM assay, an iterative method development strategy was adopted, eliminating poor performing transitions from the assay in a targeted method refinement cycle. Raw data was inspected in the Skyline software platform and the peak areas of each detected transition were normalized to that of a beta-galactosidase trypsinized peptide GDFQFNISR (SEQ ID NO:29) and an internal albumin peptide TYETTLEK (SEQ ID NO:30) to minimize run-to-run variability as a function of instrument performance. Starting with an initial 358 transitions for the ten selected peptides, multiple rounds of iterative method refinement were completed to generate the final SRM assay. The final SRM assay is a scheduled method consisting of 39 transitions with a scheduling window between two and four minutes. Reproducibility was determined via Precision Calculation of the patient screen and % cv values for each measured peptide are displayed in FIG. 16.

Data Analysis.

The methods are the same as described above in Example 1.

### Results

Sampling.

10 μl sera samples (Bioreclamation, LLC) from a control population cohort consisting of 15 healthy male and 15 female subjects were used during assay screening. To ensure that SRM results reflected only in-vivo human serum albumin micro-heterogeneities and not artificially-induced changes, a rigorous sample preparation protocol was devised and followed for every sample analyzed. See Kozikowski et al., 8 J. BIOL. SREEN 210 (2003). By storing all sera aliquots at a temperature below the threshold for biological activity, minimizing the number of freeze-thaw cycles each aliquot underwent, and minimizing the amount of time a single aliquot remained at room temperature during sample preparation, it is expected to have eliminated most potential for artificial fragmentation or sample degradation. While all potential sources of statistical variance within the selected cohort were not able to be eliminated, the 15 male and female subjects were all 20-60 years of age and each subject was paired into a male female sample set. To minimize the influence of instrument variability during quantitation, two biological replicates per sample set were analyzed by SRM-mass spectrometry for all eight selected human serum albumin biomarkers. The list of human serum albumin peptide sequences monitored, the specific fragmentation transitions, the optimized collision energy (CE), and the start/stop times in the scheduled SRM experiment are listed in FIG. 8. A signature peptide from E. coli beta-galactosidase (Sigma Aldrich, St. Louis, Mo.) was used as an external standard for determination of detection limits. An additional signature human serum albumin peptide was used to direct statistical normalization during quantitation.

Trypsin Digestion Efficiency.

The first experiments performed with the Perfinity Workstation (Perfinity Biosciences, Inc) were optimized on-column sample trypsinization using pooled sera obtained commercially (Bioreclamation, LLC). Using the pooled sera sample as a standard test matrix, UV-vis based detection of the tryptic peptide map (a c18-based linear RP-HPLC separation of the tryptic digest) was used to measure experimental digestion efficiency, reproducibility, and percent sample carry-over. To obtain the sample replicate comparison shown in FIG. 4, digestion using the pooled sera sample (Bioreclamation, LLC) was repeated five times. From this experiment, the efficiency of the digestion process was determined to be approximately 90%. This measurement was obtained by calculating the ratio between the areas under the curves of chromatographic peaks eluted during the biological gradient (5%-75%) and the areas under the curves of undigested peptide peaks eluted at high organic (75-95%). Percent carryover between sample replicates was calculated by comparing area under the curve measurements of chromatographic peaks detected during analysis using 50 μL of diluted sera to area under the curve measurements of chromatographic peaks detected with a null injection following analysis of a sera sample and end-of-run column washes described above. By incorporating two back-to-back column washes between samples, sample contamination from carry-over from previous sample injections was almost negligible (data not shown).

Limit of Detection: Retention Time and Peak Area Reproducibility.

Using the sample preparation conditions determined initially, intact E. coli beta-galctosidase (Sigma Aldrich) was serially diluted and added to a pooled control subject sera matrix (Bioreclamation, LLC). SRM analysis was performed to detect the three most abundant transitions of a signature tryptic beta-galactosidase peptide (generated during on-line sample preparation with the Perfinity Workstation). As shown in FIG. 5B, using seven serial dilutions, the limit of detection of the assay platform was determined as one femtomole. These measurements monitored the peptide GDFQFNISR MH₂⁺² ion transitions to the y₃, y₄ and b₄ fragment ions at a retention time of 2.9 minutes as shown in FIG. 5A. Retention time reproducibility for the eight diagnostic human serum albumin peptides, the signature internal standard, and the external standard included in the developed ACI assay were obtained from three technical replicates performed using the pooled control subject test sample and are shown in FIG. 7.

The Role of Gender in Trypsin Digestion.

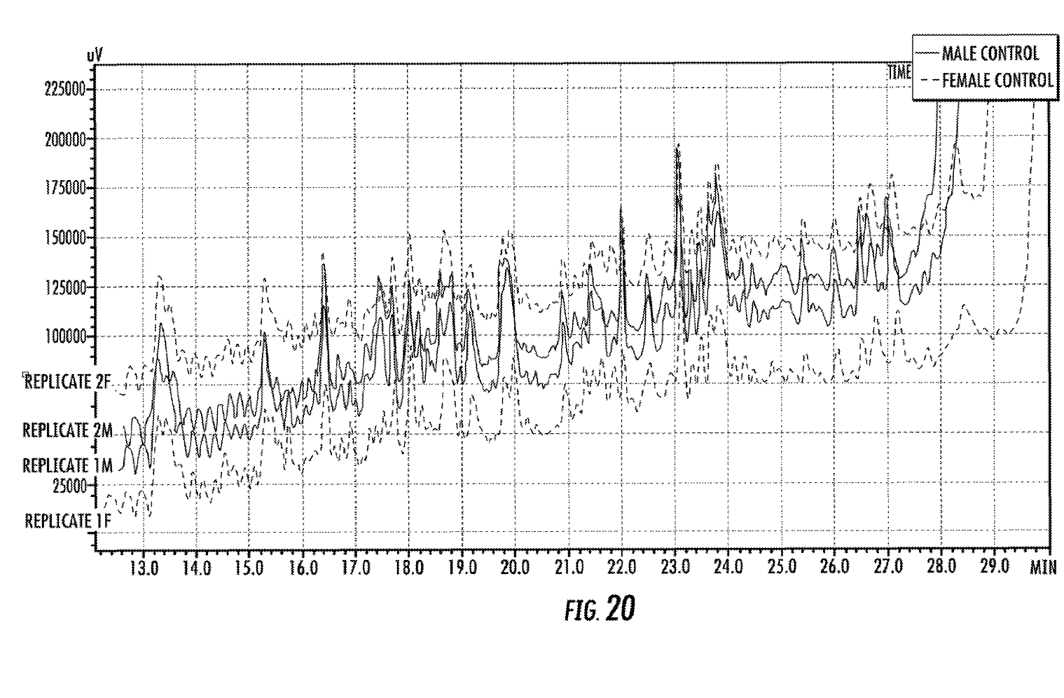

To elucidate if gender could influence digestion pattern, 5 μl from an aliquot from all 15 individual male and 15 female sera samples (Bioreclamation, LLC) were combined into two respective 75 μC sample pools. Using the sample preparation conditions determined in the first round of experimentation, the chromatographic tryptic peptide maps from both sets of pooled samples was obtained in duplicate and analyzed. While not a conclusive experiment, as shown in FIG. 5A, upon close analysis, several concrete differences do exist between the peptide maps of the pooled male and female samples. To determine if these differences were conserved across the two sample pools or if the observed differences were unique to only a few individual subjects, for all 30 patient samples, peptide maps of the digestion, desalt, and HPLC separation procedures were acquired in duplicate with the uv-vis detector of the Perfinity Workstation. As shown in FIG. 20, generally speaking, a limited degree of variability exists between the digested male and female samples shown. However, the differences between male and female subjects were largely conserved. The chromatographic peak present at particular time point was seen in a certain number of female subjects screened but absent in a certain number of screened male subjects (data not shown). While rationales for the observed differences are still not clear, such differences do have the ability to bias peptide-based diagnostic assays that incorporate digestion in front-end sample preparation.

Precision Calculations.

The mean peak areas for the eight biomarker peptides from male and female subjects are reported in FIG. 16, along with standard deviations. To assess the precision of the assay, % cvs were calculated for all eight diagnostic peptides for both male and female populations (the results of which are also included in FIG. 16). For all sixteen measurements, the % cvs were below 10% suggesting that automating front-end sample preparation increases the overall quality of the assay in addition to the speed and throughput.

Differential Expression of Selected Peptide Biomarkers as a Function of Gender.

To determine if the expression of the eight diagnostic peptides significantly differed between men and women, a two-tailed, independent t test was applied for unpaired data whereby p values <0.05 were considered significant. While the peptide maps of the two populations suggest differences during trypsin digestion that may be attributed to gender, these differences were not reflected in the mass spectrometric assay results. According to the t test analysis, the expression levels of the eight peptides were not detected at significantly different abundance in men than in women. The exact probability values (p values) generated from the performed t tests have been included in FIG. 16. FIGS. 17-19 show the box plots for all eight biomarker peptides.

### Discussion

The Perfinity Workstation represents a significant improvement in how proteomic sample preparation can be performed. By dramatically reducing the time associated with proteomic sample preparation (from 18 hour digestion to six minute digestion) while increasing the reproducibility of the process (less than 10% cvs for all detected peptides), the Perfinity Workstation increases the feasibility of SRM-based assays for clinical settings. Clinical assays need to be highly accurate, rapid, and high throughput. An SRM assay that couples mass spectrometry with the Perfinity Workstation (Perfinity Biosciences, Inc.) fulfills all three of these testing requirements. Automating sample preparation has facilitated a sera-based multiplex SRM assay that can be completed on a time-scale compatible with urgent care requirements and with the accuracy and reproducibility required by our country's current medicolegal climate.

Here, the present inventors coupled the Perfinity Workstation with mass spectrometry to successfully quantify modified human serum albumin peptides. However, one of the key advantages of this dual instrument platform is its flexibility in target selection. The instrument workflow could easily be transformed to validate other protein biomarkers, assuming no dynamic range problem exists to confound their detection. Detecting low abundant blood protein biomarkers can be challenging, if not impossible. When compared to the dynamic range of the plasma proteome (in excess of 10¹⁰), proteomic-based mass spectrometry provides a limited dynamic range—typically not exceeding 10³ in a single spectrum. Integrating alternate mass spectrometry techniques into the current instrument platform could help increase assay sensitivity and facilitate detection of lower abundant ACI target biomarker proteins. For example, assay sensitivity could be further enhanced by interfacing the QqQ with a dual stage electrodynamic ion funnel interface. Alternately, combining antibody-based enrichment with SRM-mass spectrometry could also be used to facilitate detection of lower abundant biomarker species. The Perfinity Workstation design includes two initial enrichment steps that were not incorporated into the current assay. By inserting an on-column antibody pull-down and a buffer exchange step to selectively isolate the biomarker target prior to the current protocol of on-column tryptic digestion, desalt, and separation, it may be possible to use a front-end purification strategy to address limitations caused by instrument dynamic range.

The objective of this project was to validate the feasibility of incorporating automated sample preparation into the SRM workflow. As such, analysis was restricted to a relatively small cohort of only healthy subjects. However, control population studies like the one presented here should be pre-requisites for large-scale biomarker validation efforts. By studying control subjects, it becomes possible to establish baseline population measurements for use in subsequent clinical validation studies. A positive biomarker measurement needs to be conclusively diagnostic of disease and necessarily differentiated from normal population variance. Determining the diagnostic power of any biomarker therefore requires comparative studies of the normal population. Given how gender seems to influence the onset, symptoms, and presentation of IHD (e.g. AMIs in premenopausal women are rare thus, on average, women have AMIs later in life than men, women have unrecognized AMIs more frequently than their male-counterparts, during IHD, only 30% of women experience chest-pains—the most common symptom of AMI in men, women have increased incidence of non-Q wave myocardial infarctions), it seemed especially important to establish baseline measurements of the eight selected ACI biomarker targets for both male and female control populations. More importantly, any ACI biomarker taken to market will need to be validated in clinical cohorts that include both male and female patients.

### CONCLUSION

Using SRM-mass spectrometry and automated front-end sample preparation, the present inventors have established a 35 minute, eight peptide, multiplex assay compatible with emergency department testing requirements. By replacing manual benchtop sample preparation with sample preparation performed in-line to the mass spectrometer, the model diagnostic showed a high degree of reproducibility for each of the eight target biomarker peptides when used during a high throughput screen of only control subjects. Using the automated platform, 40 samples were able to be screened a day—a dramatic improvement in throughput when compared to the sample throughput of the conventional SRM platform. Post-screen, the expression level of each targeted peptide was determined and the expression profiles between male and female subjects were compared. Statistical analysis suggests that gender does not influence the detected abundance of the eight target peptides despite observed chromatographic differences present between male and female peptide maps. Overall the present inventors have established a straightforward mass spectrometry-based platform for blood-based protein biomarker verification that can be easily adapted to study a broad range of proteins, especially those of interest to cardiac disease.

1. A method for diagnosing acute cardiac ischemia (ACI) in a patient comprising the steps of:
a. measuring the levels of one or more serum albumin biomarker peptides in a sample collected from the patient using selected reaction monitoring mass spectrometry (SRM-MS); and
b. comparing the levels of the one or more biomarkers with predefined levels of the same biomarkers that correlate to a patient having ACI and predefined levels of the same biomarkers that correlate to a patient not having ACI, wherein a correlation to one of the predefined levels provides the diagnosis.

2. The method of claim 1, wherein the one or more serum albumin biomarker peptides is selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12.

3. The method of claim 1, wherein the one or more serum albumin biomarker peptides comprises SEQ ID NO:5, SEQ ID NO:6, SEQ ID NO:7, SEQ ID NO:8, SEQ ID NO:9, SEQ ID NO:10, SEQ ID NO:11 and SEQ ID NO:12.

4. The method of claim 2, wherein the one or more serum albumin biomarker peptides further comprises one or more serum albumin biomarker peptides selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

5. The method of claim 1, wherein the one or more serum albumin biomarker peptides is selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

6. The method of claim 1, wherein the one or more serum albumin biomarker peptides comprises SEQ ID NO:5, SEQ ID NO:6, SEQ ID NO:7, SEQ ID NO:8, SEQ ID NO:9, SEQ ID NO:10, SEQ ID NO:11 and SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

7. The method of claim 1, wherein the one or more serum albumin biomarker peptides comprises SEQ ID NO:5.

8. The method of claim 1, wherein the one or more serum albumin biomarker peptides comprises SEQ ID NO:6.

9. The method of claim 1, wherein the one or more serum albumin biomarker peptides comprises SEQ ID NO:7.

10. The method of claim 1, wherein the one or more serum albumin biomarker peptides comprises SEQ ID NO:8.

11. The method of claim 1, wherein the one or more serum albumin biomarker peptides comprises SEQ ID NO:9.

12. The method of claim 1, wherein the one or more serum albumin biomarker peptides comprises SEQ ID NO:10.

13. The method of claim 1, wherein the one or more serum albumin biomarker peptides comprises SEQ ID NO:11.

14. The method of claim 1, wherein the one or more serum albumin biomarker peptides comprises SEQ ID NO:12.

15. The method of claim 1, wherein the sample is a blood, plasma, or serum sample.

16. The method of claim 15, wherein the sample is a blood sample.

17. The method of claim 15, wherein the sample is a plasma sample.

18. The method of claim 15, wherein the sample is a serum sample.

19. A method for diagnosing ACI in a patient comprising the steps of:
a. collecting a sample from the patient;
b. measuring the levels of a panel of serum albumin biomarker peptides in the sample collected from the patient using SRM-MS, wherein the panel of biomarkers comprises SEQ ID NO:5, SEQ ID NO:6, SEQ ID NO:7, SEQ ID NO:8, SEQ ID NO:9, SEQ ID NO:10, SEQ ID NO:11 and SEQ ID NO:12; and
c. comparing the levels of the panel of biomarkers with predefined levels of the same panel of biomarkers that correlate to a patient having ACI and predefined levels of the same panel of biomarkers that correlate to a patient not having ACI, wherein a correlation to one of the predefined levels provides the diagnosis.

20. The method of claim 19, wherein the panel of biomarkers further comprises one or more serum albumin biomarker peptides selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28

21. A method for determining the ACI status in a patient comprising the steps of:
a. collecting a sample from the patient;
b. measuring the levels of a panel of serum albumin biomarker peptides in the sample collected from the patient using SRM-MS, wherein the panel of biomarkers comprises SEQ ID NO:5, SEQ ID NO:6, SEQ ID NO:7, SEQ ID NO:8, SEQ ID NO:9, SEQ ID NO:10, SEQ ID NO:11 and SEQ ID NO:12; and
c. comparing the levels of the panel of biomarkers with predefined levels of the same panel of biomarkers that correlate to one or more ACI statuses selected from the group consisting of having ACI, not having ACI, progressing ACI, and regressing ACI, wherein a correlation to one of the predefined levels determines the ACI status of the patient.

22. The method of claim 21, wherein the panel of biomarkers further comprises one or more serum albumin biomarker peptides selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

23. A method for determining the ACI status in a patient comprising the steps of:
a. collecting a sample from the patient;
b. measuring the levels of a panel of serum albumin biomarker peptides in the sample collected from the patient using SRM-MS, wherein the panel of biomarkers comprises one or more modified and/or unmodified peptides from Domain I of serum albumin; and
c. comparing the levels of the panel of biomarkers with predefined levels of the same panel of biomarkers that correlate to one or more ACI statuses selected from the group consisting of having ACI, not having ACI, progressing ACI, and regressing ACI, wherein a correlation to one of the predefined levels determines the ACI status of the patient.

24. The method of claim 23, wherein the one or more peptides from Domain I of serum albumin is selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12.

25. The method of claim 23, wherein the panel of biomarkers further comprises one or more modified and/or unmodified peptides from Domain II of serum albumin.

26. The method of claim 25, wherein the one or more peptides from Domain I of serum albumin is selected from the group consisting of SEQ ID NO:21, SEQ ID NO:22, and SEQ ID NO:23.

27. The method of claim 23, wherein the panel of biomarkers further comprises one or more modified and/or unmodified peptides from Domain III of serum albumin.

28. The method of claim 27, wherein the one or more peptides from Domain I of serum albumin is selected from the group consisting of SEQ ID NO:24, SEQ ID NO:25, SEQ ID NO:26, SEQ ID NO:27, and SEQ ID NO:28.

29. A diagnostic kit for diagnosing ACI in a patient comprising:
a. a substrate for collecting a biological sample from the patient; and
b. means for measuring the levels of one or more human serum albumin biomarker peptides selected from the group consisting of SEQ ID NO:5, SEQ ID NO:6, SEQ ID NO:7, SEQ ID NO:8, SEQ ID NO:9, SEQ ID NO:10, SEQ ID NO:11 and SEQ ID NO:12.

30. The kit of claim 29, wherein the one or more human serum albumin biomarker peptides further comprises SEQ ID NO:13, SEQ ID NO:14, SEQ ID NO:15, SEQ ID NO:16, SEQ ID NO:17, SEQ ID NO:18, SEQ ID NO:19 and SEQ ID NO:20, SEQ ID NO:21, SEQ ID NO:22, SEQ ID NO:23, SEQ ID NO:24, SEQ ID NO:25, SEQ ID NO:26, SEQ ID NO:27, and SEQ ID NO:28.

31-33. (canceled)

34. A method for diagnosing ACI in a patient comprising the steps of:
a. measuring the levels of one or more modified and/or unmodified serum albumin proteins in a sample collected from the patient, wherein the modified proteins are phosphorylated and/or cysteinylated; and
b. comparing the levels of the measured one or more modified and/or unmodified serum albumin proteins with predefined levels of the same proteins that correlate to a patient having ACI and predefined levels of the same proteins that correlate to a patient not having ACI, wherein a correlation to one of the predefined levels provides the diagnosis.

35. A method for diagnosing ACI in a patient comprising the steps of:
c. measuring the levels of one or more post-translationally modified and unmodified serum albumin peptides in a sample collected from the patient using SRM-MS, wherein the post-translationally modified peptides are phosphorylated and/or cysteinylated;
d. comparing the levels of the measured one or more post-translationally modified serum albumin peptides to the levels of the measured one or more unmodified serum albumin peptides; and
e. correlating the compared levels to a patient having ACI or to a patient not having ACI, thereby providing the diagnosis.

36. A method for diagnosing ACI in a patient comprising the steps of:
f. measuring the levels of one or more post-translationally modified and unmodified serum albumin peptides in a sample collected from the patient using SRM-MS, wherein the post-translationally modified peptides are phosphorylated and/or cysteinylated;
g. comparing the ratio of the measured one or more post-translationally modified serum albumin peptides and the measured one or more unmodified serum albumin peptides to the ratio of one more modified/unmodified serum albumin peptides; and
h. correlating the compared levels to a patient having ACI or to a patient not having ACI, thereby providing the diagnosis.


SEQUENCE LISTING (30 sequences)

SEQ ID NO: 1 - protein, 609 aa, Homo sapiens [sequence omitted]

SEQ ID NO: 2 - protein, 192 aa, Homo sapiens
  MISC_FEATURE: Domain I of Human Serum Albumin
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELR

SEQ ID NO: 3 - protein, 193 aa, Homo sapiens
  MISC_FEATURE: Domain II of Human Serum Albumin
DEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKP

SEQ ID NO: 4 - protein, 198 aa, Homo sapiens
  MISC_FEATURE: Domain III of Human Serum Albumin
LVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVA

SEQ ID NO: 5 - protein, 12 aa, Homo sapiens
  MISC_FEATURE: Unmodified Human Serum Albumin Peptide from Domain I
ETYGEMADCCAK

SEQ ID NO: 6 - protein, 12 aa, Homo sapiens
  MOD_RES (10)..(10): Modified Human Serum Albumin Peptide from Domain I, tenth residue cysteinylated (cysteine, C114)
ETYGEMADCCAK

SEQ ID NO: 7 - protein, 12 aa, Homo sapiens
  MOD_RES (3)..(3): Modified Human Serum Albumin Peptide from Domain I, third residue phosphorylated (tyrosine, Y108)
ETYGEMADCCAK

SEQ ID NO: 8 - protein, 13 aa, Homo sapiens
  MISC_FEATURE: Unmodified Human Serum Albumin Peptide from Domain I
TCVADESAENCDK

SEQ ID NO: 9 - protein, 13 aa, Homo sapiens
  MOD_RES (2)..(2): Modified Human Serum Albumin Peptide from Domain I, second residue cysteinylated (cysteine, C71)
TCVADESAENCDK

SEQ ID NO: 10 - protein, 13 aa, Homo sapiens
  MOD_RES (7)..(7): Modified Human Serum Albumin Peptide from Domain I, seventh residue phosphorylated (serine, S61)
TCVADESAENCDK

SEQ ID NO: 11 - protein, 21 aa, Homo sapiens
  MISC_FEATURE: Unmodified Human Serum Albumin Peptide from Domain I
ALVLIAFAQYLQQCPFEDHVK

SEQ ID NO: 12 - protein, 21 aa, Homo sapiens
  MOD_RES (14)..(14): Modified Human Serum Albumin Peptide from Domain I, fourteenth residue cysteinylated (cysteine, C57)
ALVLIAFAQYLQQCPFEDHVK

SEQ ID NO: 13 - protein, 8 aa, Homo sapiens
  MISC_FEATURE: Unmodified Human Serum Albumin Peptide from Domain I
LCTVATLR

SEQ ID NO: 14 - protein, 8 aa, Homo sapiens
  MOD_RES (2)..(2): Modified Human Serum Albumin Peptide from Domain I, second residue cysteinylated (cysteine, C98)
LCTVATLR

SEQ ID NO: 15 - protein, 19 aa, Homo sapiens
  MISC_FEATURE: Unmodified Human Serum Albumin Peptide from Domain I
PEVDVMCTAFHDNEETFLK

SEQ ID NO: 16 - protein, 19 aa, Homo sapiens
  MOD_RES (7)..(7): Modified Human Serum Albumin Peptide from Domain I, seventh residue cysteinylated (cysteine, C200)
PEVDVMCTAFHDNEETFLK

SEQ ID NO: 17 - protein, 7 aa, Homo sapiens
  MISC_FEATURE: Unmodified Human Serum Albumin Peptide from Domain I
YLYEIAR

SEQ ID NO: 18 - protein, 7 aa, Homo sapiens
  MOD_RES (3)..(3): Modified Human Serum Albumin Peptide from Domain I, second residue phosphorylated (tyrosine, Y163)
YLYEIAR

SEQ ID NO: 19 - protein, 7 aa, Homo sapiens
  MISC_FEATURE: Unmodified Human Serum Albumin Peptide from Domain I
AACLLPK

SEQ ID NO: 20 - protein, 7 aa, Homo sapiens
  MOD_RES (3)..(3): Modified Human Serum Albumin Peptide from Domain I, third residue cysteinylated (cysteine, C200)
AACLLPK

SEQ ID NO: 21 - protein, 12 aa, Homo sapiens
  MISC_FEATURE: Unmodified Human Serum Albumin Peptide from Domain II
YICENQDSISSK

SEQ ID NO: 22 - protein, 12 aa, Homo sapiens
  MOD_RES (8)..(8): Modified Human Serum Albumin Peptide from Domain II, eighth residue phosphorylated (serine, S293)
YICENQDSISSK

SEQ ID NO: 23 - protein, 12 aa, Homo sapiens
  MOD_RES (10)..(10): Modified Human Serum Albumin Peptide from Domain II, tenth residue phosphorylated (serine, S295)
YICENQDSISSK

SEQ ID NO: 24 - protein, 14 aa, Homo sapiens
  MISC_FEATURE: Unmodified Human Serum Albumin Peptide from Domain III
VPQVSTPTLVEVSR

SEQ ID NO: 25 - protein, 14 aa, Homo sapiens
  MOD_RES (8)..(8): Modified Human Serum Albumin Peptide from Domain III, eighth residue phosphorylated (threonine, T445)
VPQVSTPTLVEVSR

SEQ ID NO: 26 - protein, 14 aa, Homo sapiens
  MOD_RES (6)..(6): Modified Human Serum Albumin Peptide from Domain III, sixth residue phosphorylated (threonine, T443)
VPQVSTPTLVEVSR

SEQ ID NO: 27 - protein, 14 aa, Homo sapiens
  MOD_RES (5)..(5): Modified Human Serum Albumin Peptide from Domain III, fifth residue phosphorylated (serine, S442)
VPQVSTPTLVEVSR

SEQ ID NO: 28 - protein, 14 aa, Homo sapiens
  MOD_RES (5)..(6): Modified Human Serum Albumin Peptide from Domain III, fifth and sixth residues phosphorylated (serine, S442 and threonine, T443))
VPQVSTPTLVEVSR

SEQ ID NO: 29 - protein, 9 aa, Escherichia coli
  MISC_FEATURE: Peptide from E. coli beta-galactosidase, external control
GDFQFNISR

SEQ ID NO: 30 - protein, 8 aa, Homo sapiens
  MISC_FEATURE: Unmodified Human Serum Albumin Peptide, internal standard
TYETTLEK